(19) United States
(12) Patent Application Publication
(10) Pub. No.: US 2009/0258096 A1
(43) Pub. Date: Oct. 15, 2009

(54) Anticancer Methods Employing Extracts of Gleditsia sinensis Lam

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: BioNovo, Inc., Emeryville, CA (US)

(21) Appl. No.: 12/422,113

(22) Filed: Apr. 10, 2009

Related U.S. Application Data
(60) Provisional application No. 61/044,396, filed on Apr. 11, 2008.

(51) Int. Cl. A61K 36/483; A61P 35/00

(52) U.S. Cl. 424/769

(57) ABSTRACT

Selective apoptotic extracts of Gleditsia sinensis Lam are provided. Also provided are methods of using said extracts to induce apoptosis in specific cells, especially in a human. Provided as well are uses of the extracts of Gleditsia sinensis Lam for the preparation of a medicament for the selective induction of apoptosis.

### PRIORITY

The present application claims priority under 35 U.S.C. § 119(e) from U.S. provisional patent application 61/044,396, filed Apr. 11, 2008, the entire contents of which are incorporated herein by reference.

### FIELD OF THE INVENTION

The present invention relates to plant extract compositions, and more particularly to compositions comprising extracts of plant species belonging to the species Gleditsia sinensis Lam. The invention further relates to methods of using and methods of making such plant extract compositions.

### BACKGROUND

A hallmark feature of cancerous cells is uncontrolled proliferation. Among the causes of uncontrolled proliferation that have been identified, an apparently important one is resistance to the process of programmed cell death, also known as apoptosis. Apoptosis is a process multicellular organisms employ to prevent uncontrolled cell proliferation and to eliminate cells that have become sick, malignant, or superfluous. The process of apoptosis involves a multi-step cascade in which cells are degraded from within through the concerted action of proteolytic enzymes and DNA endonucleases, resulting in the formation of apoptotic bodies that are then removed by scavenger cells. Research to date has shown that much of the intracellular degradation is carried out through the action of the caspases, a family of proteolytic enzymes that cleave adjacent to aspartate residues.

Despite recent advances in breast cancer treatments, current treatment regimes often lead to toxic (sometimes treatment-limiting) side effects. Moreover, current treatments are mostly ineffective against metastatic breast cancer. While early screening and treatment can improve prognosis for many patients, such screening is not uniform and some cancers propagate too quickly to be detected in an early stage by routine screening. There remains a need for treatment options that are less toxic, active against later-stage cancers or both.

One particularly treatment-refractive type of cancer is estrogen receptor negative breast cancer. All the currently approved treatments for breast cancer in the United States are most effective against estrogen receptor positive cancer. A breast cancer may begin in estrogen receptor negative tissue, or may cease to express estrogen receptor as an adaptive response to cancer therapy. For patients with estrogen receptor negative breast cancer, the options are few. Thus there is a need for treatment options for those patients whose breast cancer is estrogen receptor negative.

A sub-class of estrogen receptor negative cancer is breast cancer that is negative for the estrogen receptor (ER) as well as one or both of the progesterone receptor (PR) and/or human epidermal growth factor 2 (Her2/neu). A particularly treatment-refractory subset of this sub-class of ER negative cancers are the so-called “triple negative” breast cancers—i.e. those that are negative for ER, PR and Her2/neu. For those patients with triple negative breast cancer, treatment options are very limited. Thus there is a present need for treatment options for this group of patients.

Treatment-refractory cancers, especially breast cancers, are unfortunately common. Once a patient has undergone one or more treatment regimens for cancer, their options for further treatment for cancer become more limited and potentially more toxic. There is thus a need for options for patients who have undergone one or more previous rounds of treatment for cancer, but whose cancer has not responded, or has ceased to respond, to treatment.

The foregoing and other needs are addressed by embodiments of the invention, as described in more detail in the following disclosure, including the attached claims and drawings.

### SUMMARY OF THE INVENTION

Some embodiments of the invention provide a method of treating a patient having estrogen receptor (ER) negative breast cancer, comprising administering a therapeutically effective amount of an extract of Gleditsia sinensis Lam effective to the patient. In some embodiments, the therapeutically effective amount of the extract of Gleditsia sinensis Lam is about 0.001 to about 100 grams dry weight of the extract per day. In some embodiments, the therapeutically effective amount of the extract of Gleditsia sinensis Lam is about 0.001 to about 10 grams dry weight of the extract per day. In some embodiments, the therapeutically effective amount of the extract of Gleditsia sinensis Lam is about 1-100 grams dry weight of the extract per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the extract of Gleditsia sinensis Lam is in an oral dosage form. In some embodiments, the oral dosage form is an elixir, a powder, one or more tablets, or one or more capsules.

Some embodiments of the invention provide a pharmaceutical composition comprising a therapeutically effective amount of an extract of Gleditsia sinensis Lam, wherein the therapeutically effective amount is effective to treat estrogen receptor (ER) negative breast cancer. In some embodiments, the therapeutically effective amount of the extract of Gleditsia sinensis Lam is about 0.001 to about 100 grams dry weight of the extract per day. In some embodiments, the therapeutically effective amount of the extract of Gleditsia sinensis Lam is about 0.001 to about 10 grams dry weight of the extract per day. In some embodiments, the therapeutically effective amount of the extract of Gleditsia sinensis Lam is about 1-100 grams dry weight of the extract per day. In some embodiments, the ER negative cancer is estrogen receptor alpha (ERα) negative. In some embodiments, the ER negative breast cancer is also negative for one or both of progesterone receptor (PR) and/or Her2/neu. In some embodiments, the ER negative cancer is triple negative breast cancer. In some embodiments, the cancer is metastatic. In some embodiments, the extract of Gleditsia sinensis Lam is in an oral dosage form. In some embodiments, the oral dosage form is an elixir, a powder, one or more tablets, or one or more capsules.

Some embodiments of the invention provide a medicament for treatment of estrogen receptor (ER) negative breast cancer comprising a therapeutically effective amount of an extract of Gleditsia sinensis Lam. In some embodiments, the therapeutically effective amount of the extract of Gleditsia sinensis Lam is about 0.001 to about 100 grams dry weight of the extract per day. In some embodiments, the therapeutically effective amount of the extract of Gleditsia sinensis Lam is about 0.001 to about 10 grams dry weight of the extract per day. In some embodiments, the therapeutically effective amount of the extract of Gleditsia sinensis Lam is about 1-100 grams dry weight of the extract per day. In some embodiments, the cancer is estrogen receptor alpha (ERα) negative. In some embodiments, the ER negative breast cancer is triple negative breast cancer. In some embodiments, the ER negative breast cancer is also negative for one or both of progesterone receptor (PR) and/or Her2/neu. In some embodiments, the ER negative breast cancer is metastatic. In some embodiments, the extract of Gleditsia sinensis Lam is in an oral dosage form. In some embodiments, the oral dosage form is an elixir, a powder, one or more tablets, or one or more capsules.

Some embodiments of the invention provide a use of an extract of Gleditsia sinensis Lam for preparation of a medicament for treatment of an estrogen receptor (ER) negative breast cancer. In some embodiments, the therapeutically effective amount of the extract of Gleditsia sinensis Lam is about 0.001 to about 100 grams dry weight of the extract per day. In some embodiments, the therapeutically effective amount of the extract of Gleditsia sinensis Lam is about 0.001 to about 10 grams dry weight of the extract per day. In some embodiments, the therapeutically effective amount of the extract of Gleditsia sinensis Lam is about 1-100 grams dry weight of the extract per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the extract of Gleditsia sinensis Lam is in an oral dosage form. In some embodiments, the oral dosage form is an elixir, a powder, one or more tablets, or one or more capsules.

Some embodiments of the invention provide a method of treating a patient having cancer that does not express an estrogen receptor (ER), comprising administering a therapeutically effective amount of an extract of Gleditsia sinensis Lam effective to the patient. In some embodiments, the therapeutically effective amount of the extract of Gleditsia sinensis Lam is about 0.001 to about 100 grams dry weight of the extract per day. In some embodiments, the extract of Gleditsia sinensis Lam is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a pharmaceutical composition comprising a therapeutically effective amount of an extract of Gleditsia sinensis Lam, wherein the therapeutically effective amount is effective to treat a cancer that does not express an estrogen receptor (ER). In some embodiments, the therapeutically effective amount of the extract of Gleditsia sinensis Lam is about 0.001 to about 100 grams dry weight of the extract per day. In some embodiments, the extract of Gleditsia sinensis Lam is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a medicament for treatment of cancer that does not express an estrogen receptor (ER) comprising a therapeutically effective amount of an extract of Gleditsia sinensis Lam. In some embodiments, the therapeutically effective amount of the extract of Gleditsia sinensis Lam is about 0.001 to about 100 grams dry weight of the extract per day. In some embodiments, the extract of Gleditsia sinensis Lam is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a use of an extract of Gleditsia sinensis Lam for preparation of a medicament for the treatment of a cancer that does not express an estrogen receptor (ER). In some embodiments, the therapeutically effective amount of the extract of Gleditsia sinensis Lam is about 0.001 to about 100 grams dry weight of the extract per day. In some embodiments, the extract of Gleditsia sinensis Lam is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a method of treating a patient having estrogen receptor (ER) negative breast cancer, comprising administering a therapeutically effective amount of oleanolic acid, or a pharmaceutically acceptable salt or derivative thereof, to the patient. In some embodiments, the therapeutically effective amount of oleanolic acid is about 0.001 to about 100 grams per day. In some embodiments, the therapeutically effective amount of oleanolic acid is about 0.001 to about 10 grams per day. In some embodiments, the therapeutically effective amount of oleanolic acid is about 1-100 grams per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the oleanolic acid, or pharmaceutically acceptable salt or derivative thereof, is in an oral dosage form. In some embodiments, the oral dosage form is an elixir, a powder, one or more tablets, or one or more capsules.

Some embodiments of the invention provide a pharmaceutical composition comprising a therapeutically effective amount of oleanolic acid, wherein the therapeutically effective amount is effective to treat estrogen receptor (ER) negative breast cancer. In some embodiments, the therapeutically effective amount of oleanolic acid is about 0.001 to about 100 grams per day. In some embodiments, the therapeutically effective amount of oleanolic acid is about 0.001 to about 10 grams per day. In some embodiments, the therapeutically effective amount of oleanolic acid is about 1-100 grams per day. In some embodiments, the ER negative cancer is estrogen receptor alpha (ERα) negative. In some embodiments, the ER negative breast cancer is also negative for one or both of progesterone receptor (PR) and/or Her2/neu. In some embodiments, the ER negative cancer is triple negative breast cancer. In some embodiments, the cancer is metastatic. In some embodiments, the oleanolic acid, or pharmaceutically acceptable salt or derivative thereof, is in an oral dosage form. In some embodiments, the oral dosage form is an elixir, a powder, one or more tablets, or one or more capsules.

Some embodiments of the invention provide a medicament for treatment of estrogen receptor (ER) negative breast cancer comprising a therapeutically effective amount of oleanolic acid, or a pharmaceutically acceptable salt or derivative thereof. In some embodiments, the therapeutically effective amount of oleanolic acid is about 0.001 to about 100 grams per day. In some embodiments, the therapeutically effective amount of oleanolic acid is about 0.001 to about 10 grams per day. In some embodiments, the therapeutically effective amount of oleanolic acid is about 1-100 grams per day. In some embodiments, the cancer is estrogen receptor alpha (ERα) negative. In some embodiments, the ER negative breast cancer is triple negative breast cancer. In some embodiments, the ER negative breast cancer is also negative for one or both of progesterone receptor (PR) and/or Her2/neu. In some embodiments, the ER negative breast cancer is metastatic. In some embodiments, the oleanolic acid, or pharmaceutically acceptable salt or derivative thereof, is in an oral dosage form. In some embodiments, the oral dosage form is an elixir, a powder, one or more tablets, or one or more capsules.

Some embodiments of the invention provide a use of a composition comprising a therapeutically effective amount of oleanolic acid, or a pharmaceutically acceptable salt or derivative thereof, for preparation of a medicament for treatment of an estrogen receptor (ER) negative breast cancer. In some embodiments, the therapeutically effective amount of oleanolic acid is about 0.001 to about 100 grams per day. In some embodiments, the therapeutically effective amount of oleanolic acid is about 0.001 to about 10 grams per day. In some embodiments, the therapeutically effective amount of oleanolic acid is about 1-100 grams per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the oleanolic acid, or pharmaceutically acceptable salt or derivative thereof, is in an oral dosage form. In some embodiments, the oral dosage form is an elixir, a powder, one or more tablets, or one or more capsules.

Some embodiments of the invention provide a method of treating a patient having cancer that does not express an estrogen receptor (ER), comprising administering a therapeutically effective amount of oleanolic acid, or a pharmaceutically acceptable salt or derivative thereof, to the patient. In some embodiments, the therapeutically effective amount of oleanolic acid is about 0.001 to about 100 grams per day. In some embodiments, the oleanolic acid, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a pharmaceutical composition comprising a therapeutically effective amount of oleanolic acid, or a pharmaceutically acceptable salt or derivative thereof, wherein the therapeutically effective amount is effective to treat a cancer that does not express an estrogen receptor (ER). In some embodiments, the therapeutically effective amount of oleanolic acid is about 0.001 to about 100 grams per day. In some embodiments, the oleanolic acid, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a medicament for treatment of cancer that does not express an estrogen receptor (ER), comprising a therapeutically effective amount of oleanolic acid, or a pharmaceutically acceptable salt or derivative thereof. In some embodiments, the therapeutically effective amount of oleanolic acid is about 0.001 to about 100 grams per day. In some embodiments, the oleanolic acid, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a use of oleanolic acid, or a pharmaceutically acceptable salt or derivative thereof, for preparation of a medicament for the treatment of a cancer that does not express an estrogen receptor (ER), comprising a therapeutically effective amount of oleanolic acid. In some embodiments, the therapeutically effective amount of oleanolic acid is about 0.001 to about 100 grams per day. In some embodiments, the oleanolic acid, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a method of treating a patient having estrogen receptor (ER) negative breast cancer, comprising administering a therapeutically effective amount of at least one saponin, or a pharmaceutically acceptable salt thereof, to the patient, wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, and mTORC2 activity in vitro. In some embodiments, the saponin posses mTORC1, mTORC2, and Akt inhibitory activity, and disrupts lipid rafts (LRs) in vitro. In some embodiments, the therapeutically effective amount of the saponin is about 0.001 to about 100 grams per day. In some embodiments, the therapeutically effective amount of the saponin is about 0.001 to about 10 grams per day. In some embodiments, the therapeutically effective amount of the saponin is about 1-100 grams per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the saponin, is in an oral dosage form. In some embodiments, the oral dosage form is an elixir, a powder, one or more tablets, or one or more capsules.

Some embodiments of the invention provide a pharmaceutical composition comprising a therapeutically effective amount of at least one saponin, or a pharmaceutically acceptable salt thereof, wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, mTORC2 and Akt inhibitory activity in vitro. In some embodiments, the saponin posses mTORC1, mTORC2, Akt inhibitory activity, and disrupts lipid rafts (LRs) in vitro. In some embodiments, the therapeutically effective amount of the saponin is about 0.001 to about 100 grams per day. In some embodiments, the therapeutically effective amount of the saponin is about 0.001 to about 10 grams per day. In some embodiments, the therapeutically effective amount of the saponin is about 1-100 grams per day. In some embodiments, the ER negative cancer is estrogen receptor alpha (ERα) negative. In some embodiments, the ER negative breast cancer is also negative for one or both of progesterone receptor (PR) and/or Her2/neu. In some embodiments, the ER negative cancer is triple negative breast cancer. In some embodiments, the cancer is metastatic. In some embodiments, the saponin is in an oral dosage form. In some embodiments, the oral dosage form is an elixir, a powder, one or more tablets, or one or more capsules.

Some embodiments of the invention provide a medicament for treatment of estrogen receptor (ER) negative breast cancer, comprising a therapeutically effective amount of saponin, or a pharmaceutically acceptable salt thereof, wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, mTORC2 and Akt inhibitory activity in vitro. In some embodiments, the saponin posses mTORC1, mTORC2, Akt inhibitory activity, and disrupts lipid rafts (LRs) in vitro. In some embodiments, the therapeutically effective amount of the saponin is about 0.001 to about 100 grams per day. In some embodiments, the therapeutically effective amount of the saponin is about 0.001 to about 10 grams per day. In some embodiments, the therapeutically effective amount of the saponin is about 1-100 grams per day. In some embodiments, the cancer is estrogen receptor alpha (ERα) negative. In some embodiments, the ER negative breast cancer is triple negative breast cancer. In some embodiments, the ER negative breast cancer is also negative for one or both of progesterone receptor (PR) and/or Her2/neu. In some embodiments, the ER negative breast cancer is metastatic. In some embodiments, the saponin, or pharmaceutically acceptable salt or derivative thereof, is in an oral dosage form. In some embodiments, the oral dosage form is an elixir, a powder, one or more tablets, or one or more capsules.

Some embodiments of the invention provide a use of a therapeutically effective amount of a saponin for preparation of a medicament for treatment of an estrogen receptor (ER) negative breast cancer, wherein the saponin possesses mTORC1, mTORC2, and/or possesses Akt inhibitory activity, or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, mTORC2 and Akt inhibitory activity in vitro. In some embodiments, the saponin posses mTORC1, mTORC2, Akt inhibitory activity, and disrupts lipid rafts (LRs) in vitro. In some embodiments, the therapeutically effective amount of the saponin is about 0.001 to about 100 grams per day. In some embodiments, the therapeutically effective amount of the saponin is about 0.001 to about 10 grams per day. In some embodiments, the therapeutically effective amount of the saponin is about 1-100 grams per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the saponin, or pharmaceutically acceptable salt or derivative thereof, is in an oral dosage form. In some embodiments, the oral dosage form is an elixir, a powder, one or more tablets, or one or more capsules.

Some embodiments of the invention provide a method of treating a patient having cancer that does not express an estrogen receptor (ER), comprising administering a therapeutically effective amount of a saponin to the patient, wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, and mTORC2 activity in vitro. In some embodiments, the saponin posses mTORC1, mTORC2, and Akt inhibitory activity, and disrupts lipid rafts (LRs) in vitro. In some embodiments, the therapeutically effective amount of the saponin is about 0.001 to about 100 grams per day. In some embodiments, the saponin, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a pharmaceutical composition comprising a therapeutically effective amount of a saponin, wherein the therapeutically effective amount is effective to treat a cancer that does not express an estrogen receptor (ER), and wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, and mTORC2 activity in vitro. In some embodiments, the saponin posses mTORC1, mTORC2, and Akt inhibitory activity, and disrupts lipid rafts (LRs) in vitro. In some embodiments, the therapeutically effective amount of the saponin is about 0.001 to about 100 grams per day. In some embodiments, the saponin, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a medicament for treatment of cancer that does not express an estrogen receptor (ER), comprising a therapeutically effective amount of a saponin, wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, and mTORC2 activity in vitro. In some embodiments, the saponin posses mTORC1, mTORC2, and Akt inhibitory activity, and disrupts lipid rafts (LRs) in vitro.

The medicament of claim 191, wherein the therapeutically effective amount of the saponin is about 0.001 to about 100 grams per day. In some embodiments, the saponin, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Some embodiments of the invention provide a use of a saponin for preparation of a medicament for the treatment of a cancer that does not express an estrogen receptor (ER), wherein the saponin possesses mTORC1, mTORC2, and/or possesses Akt inhibitory activity, and/or disrupts lipid rafts (LRs) in vitro. In some embodiments, the saponin possesses mTORC1 and mTORC2 activity. In some embodiments, the saponin possesses Akt inhibitory activity. In some embodiments, the saponin disrupts lipid rafts. In some embodiments, the saponin possesses mTORC1, and mTORC2 activity in vitro. In some embodiments, the saponin posses mTORC1, mTORC2, and Akt inhibitory activity, and disrupts lipid rafts (LRs) in vitro. In some embodiments, the therapeutically effective amount of the saponin is about 0.001 to about 100 grams per day. In some embodiments, the saponin, or pharmaceutically acceptable salt or derivative thereof, is in an oral dosage form. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D show that a composition comprising 0.090 mg/mL of a dried extract of Gleditsia sinensis Lam (BN107) induces apoptosis in breast cancer cell lines but not in transformed and normal cells and cell lines. 1A. Annexin V-PI staining. 1B. DNA fragmentation. 1C. Cytochrome C release. 1D. Activation of caspases 3 and 9. If not otherwise indicated, HS578T or MDA-MB-231 cells were treated with BN107 (90 μg/mL of solid extract of the fruit of Gleditsia sinensis Lam.

FIGS. 2A-2C show that ERα expression rescues BN107-induced apoptosis. 2A: MDA-MB231 cells infected with LacZ or Era virus were treated with BN107 in the presence of estrogen (10 nM) and analyzed with Annexin/PI binding. 2B Western analysis of ERα expression. 2C: Real time 1 RTPCR analysis of WISP2 expression, a downstream target of ERα.

FIG. 3 is a western blot depicting an analysis of proteins involved in selected signaling and cellular pathways. Hs578T and MCF7 cells were treated with BN107 and harvested at the indicated time points.

FIG. 4: BN107 induces apoptosis via mitochondrial machinery in ER− breast cancer cells, assessed by 5A. Percent survival cells (AnnexinV−, PI−) in various cell lines, 5B. mitochondrial transmembrane potential assessed by JC-1 staining, 5C. Activation of caspases 3 and 9, D. Western blot showing Cytochrome C released in cytosol. Cells were treated with BN107 (70 μg/ml) and harvested after 18 (A), 6 (B), and 3 (C) hours of treatment.

FIG. 5: ERα expression rescues MDA-MB-231 cells from BN107 induced apoptosis. 5A. Western blot showing ERα expression in MDA-MB231 cells infected with LacZ or ERα virus. 5B. Cells were treated with BN107 for 18 hrs in the presence of 10 nM estrogen and analyzed with Annexin/PI binding. The chart shows percentage of Annexin−PI−(live) cells. 5C. MDA-MB-231 cells were treated with a differentiating histone deacetylase inhibitor, tricostatin A (TsA, 50 nM) or DMSO for 2 days. The cells were then treated with BN107 and analyzed with Annexin/PI binding as in B.

FIG. 6: Induction of ROS or activation of p38 pathway in ER− breast cancer cells is not the primary cause of apoptosis induced by BN107. 6A. ROS production was measured using ROS-sensitive probe CM-H2DCFDA. Chart shows mean FL1 fluorescence. 6B. Percent survival cells in Hs578T cells pretreated with strong ROS scavengers, 10 mM NAC or 50 μM BHT, followed by BN107 treatment for 18 hours. 6C. Western blot showing levels of phosphorylated- p38 and Erk. 6D. Percent survival cells in Hs578T cells pre-treated with 20 μM p38 antagonist, SB202190, followed by BN107 treatment for 18 hours.

FIG. 7: Cholesterol depletion in the LRs is potentially the mechanism mediating the pro-apoptotic effect of BN107 in ER− breast cancer cells. 7A. Percent survival cells in Hs578T cells pretreated with 50 μM isoprenoid precursors, FOH or GGOH, followed by treatment with BN107 for 18 hours. 7B. Cholesterol content in sucrose-density fractions collected from MDA-MB-231 or MCF7 cells treated with BN107 for 4 hours. LR, lipid rafts (Fractions 3-5), non-LR plasma membrane (Fractions 6). 7C. Percent survival cells in Hs578T cells treated with 70 μg/ml BN107, 0.2 mg/ml BZL101, or 500 nM taxol alone or with 500 μM cholesterol (CHL) for 18 hours.

FIG. 8: LRs proteins and LR-mediated mTORC1 and mTORC2 signalings are disrupted by BN107 or oleanolic acid treatment. 8A. Immunofluorescence staining of caveolin 1 (CAV1) and CD44 (green) in MDA-MB-231 cells. 8B. Dot plot analysis of GM-1. Western analysis of LRs, non-LR plasma membrane, and cytosolic fractions (C), and total cellular lysate (D). All cells were treated with 70 μg/ml BN107 or 110 μM oleanolic acid (±500 μM CHL) for 4 hr. Fractions were spotted directly from fractions (B), or were precipitated to load the same amount of protein (C).

### DETAILED DESCRIPTION OF THE INVENTION

Disclosed herein are pharmaceutical compositions comprising inter alia an extract of the taxonomic species of plant referred to as Gleditsia sinensis Lam. Further embodiments disclosed herein provide selectively apoptotic methods of using the herein-described compositions. The selectively apoptotic compositions described herein possess the activity of inducing apoptosis in abnormally dividing cells, such as cancer cells, while not disturbing the normal cellular processes of normal calls. While not desiring to be limited by theory, it is believed that the active ingredients in the disclosed pharmaceutical compositions act through the caspase pathway to induce apoptosis in cells that have otherwise lost their ability to self-regulate through the process of apoptosis. Such active ingredients, which are extracted from Gleditsia sinensis Lam, especially the fruit thereof, inhibit the activity of AKT (a serine/threonine protein kinase) and mTOR kinases in cancer cell, thereby suggesting their activity in inducing or restoring apoptosis in cancerous cells.

Treatment of breast cancer cells with aqueous extract of Gleditsia sinensis Lam (0.5 mg of the dried, solid extract per mL of aqueous solution) induces significant cell death in many of the cancer cell lines. Normal mammary epithelial cells and fibroblasts are resistant to the cytotoxic effects of the Gleditsia sinensis Lam extract. Breast cancer cells that were sensitive to the Gleditsia sinensis Lam extract underwent apoptotic cell death (confirmed by DNA fragmentation, caspase activation, cleavage of PARP and Annexin V staining). In addition to caspase 3, it was observed that activation of caspases 4 and 9, which are linked to apoptosis induced by endoplasmic reticulum stress, was also induced by the 0.090 mg/mL aqueous extract of Gleditsia sinensis Lam. This solution induced rapid inactivation of AKT and mTOR kinases in breast cancer, but not in non-transformed cells. Expression of several genes that have well-known pro-apoptotic and anti-proliferative characteristics was also induced by the aqueous extract of Gleditsia sinensis Lam. It is thus an aspect of the invention to take advantage of the selective pro-apoptotic effects of extracts of Gleditsia sinensis Lam for the treatment of multicellular organisms, such as mammals, and in particular humans.

Extracts of Gleditsia sinensis Lam

The active ingredients employed in the pharmaceutical compositions, medicaments, uses (processes) for manufacturing medicaments and methods of treating cancer, such as ER negative breast cancer, comprise extracts of Gleditsia sinensis Lam or an apoptotically active component thereof. In some embodiments, the active ingredients consist essentially of Gleditsia sinensis Lam or an apoptotically active component thereof. In some embodiments, the active ingredients consist of Gleditsia sinensis Lam or an apoptotically active component thereof.

An “extract” is a solution, concentrate or residue (dried extract solution) that results when a plant part is contacted with an extraction solvent under conditions suitable for one or more compounds from the plant to partition from the plant matter into the extraction solvent; the solution is then optionally reduced in volume to form a concentrate or a residue.

Suitable extraction media for the present invention include water and ethyl alcohol. Specifically, where water is the extraction solvent, purified water is suitable. Purified water includes distilled water, deionized water, water for injection, ultrafiltered water, and other forms purified of water. Ethyl alcohol that is employed in some embodiments of the invention is grain ethanol, and in particular undenatured ethanol (e.g. pure grain ethanol, optionally containing some water, e.g. up to about 10% water). In some embodiments, the extraction solvent is water, ethanol, or a mixture thereof. A concentrate or residue may be prepared by reducing (e.g. evaporating or lyophilizing) the extraction solution. Whether in the original extraction solvent, reduced concentrate, or residue form, each of these preparations is considered an “extract” for the purposes of the invention.

A method of producing the plant extract according to the invention optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

The extraction medium (solvent) is then contacted with the plant matter under conditions suitable for causing one or more phytochemicals, in particular selectively apoptotic phytochemicals, to partition from the plant matter into the extraction medium. (Apoptotic components of an extract of Gleditsia sinensis Lam include apoptotic phytochemicals, such as oleanolic acid.) Such conditions include, in some cases, heating the extraction medium to a temperature above room temperature, agitation, contact time, etc. Exemplary temperatures for extraction are from about 50° C. to the boiling point of the extraction solvent. Where water is the extraction solvent, the extraction temperature is generally from room temperature to about 100° C.; temperatures of from about 50° C. to about 80° C. are especially suitable, and temperatures of about 75° C. are particularly suitable. In the case of ethanol as an extraction solvent, the extraction temperature is generally from about room temperature to about 78.5° C.; temperatures of from about 50° C. to about 78° C. are especially suitable and a temperature of about 75° C. is particularly suitable. The person of skill in the art will recognize that the proper balance should be drawn between extraction efficiency on the one hand and phytochemical compound stability on the other.

Once the extraction medium and the plant matter are combined, they are optionally agitated to ensure efficient exchange of selectively apoptotic compound from the plant matter into the extraction medium, and are left in contact for a time sufficient to extract a useful amount of apoptotic phytochemical compound from the plant matter into the extraction medium. After such time has elapsed (e.g. from about 5 min. to about 10 hr., more particularly from about 10 min. to about 5 hr., especially about 30 min. to about 2 hr.), the extraction medium containing the apoptotic phytochemical compound or compounds is separated from the plant matter. Such separation is accomplished by an art-recognized method, e.g. by filtration, decanting, etc.

A composition according to the invention includes an herein-described plant extract or a composition comprising an herein-described plant extract of the invention. In such embodiments, the herein-described composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the herein-described plant extract.

Gleditsia sinensis Lam

The species Gleditsia sinensis Lam is a deciduous tree growing to 12 m at a medium rate. The flowers are hermaphroditic, have both male and female organs, and are pollinated by insects. The plant is known to fix nitrogen. The plant prefers light (sandy), medium (loamy) and heavy (clay) soils and requires well-drained soil. The plant prefers acid, neutral and basic (alkaline) soils. It cannot grow in the shade. It requires dry or moist soil and can tolerate drought. It can tolerate atmospheric pollution. Trees have a light canopy; they come into leaf late in the spring and drop their leaves in early autumn.

Preparation of Extract

In particular embodiments, fruit are harvested from the tree and contacted with the extraction medium within a short period after harvesting. The extraction medium is a suitable liquid solvent, e.g. ethyl acetate, water or ethanol. The extraction medium is in some cases ethyl acetate, water, ethanol or another relatively polar liquid solvent. In some cases, the extraction medium is either diluted or reduced. The extraction medium may be fully reduced, whereby the extract takes the form of a residue (residual extract). Thus, the extract contains at a minimum one or more plant-derived compounds (phytochemicals), optionally dissolved in a solvent, which are drawn into the extraction medium through one or more steps of contacting the extraction medium and the plant or plant parts. A concentrated or residual extract may be reconstituted by adding a suitable diluent, e.g. ethyl acetate, water and/or ethanol, to form a reconstituted extract.

In some embodiments, compositions comprising plant extracts include pure extracts or partitioned extracts (including extracts in which one or more selectively apoptotic active compounds in the extract have been enriched) and combinations of such extracts with one or more additional ingredients. In some embodiments, the compositions include those in a variety of physical forms, including solid, semi-solid, liquid, colloidal, etc. Where the compositions are intended for pharmaceutical use, the additional ingredients are pharmaceutically acceptable. Where the compositions according to the invention are intended for use in assays or other uses that are not directed toward a living body, the additional ingredient(s) may be either pharmaceutically acceptable or not.

In some embodiments, a pure extract may be combined with one or more organic solvents. Such organic solvents may be of various polarities. In some embodiments, suitable solvents include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

In some embodiments, the compositions comprise pure extracts or combinations of extracts with one or more additional solvents. In some embodiments, the extract includes a partitioned or further purified extract. Partitioning or purification may be conducted using various separation techniques, including chromatography. In some embodiments, the extract is a purified or partitioned extract obtained by means of anion exchange chromatography, cation exchange chromatography, reverse phase chromatography, normal phase chromatography, affinity chromatography or exclusion chromatography, to further concentrate active agents in the extract. In some embodiments, the purified or partitioned extract is obtained via one or more steps of liquid chromatography, such as high performance liquid chromatography (HPLC). In some embodiments, high performance liquid chromatography is preparative scale high performance liquid chromatography. In some embodiments, the HPLC is reverse phase or ion exchange chromatography. Other means of separation may also be used to purify or partition the extract, including separation in a separatory funnel or other bi- or multi-phasic separatory mechanism. In some embodiments, the purified or partitioned extract may be combined with one or more additional active or inactive ingredients, such as solvents, diluents, etc. In some embodiments, suitable solvents may include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

Suitable additional ingredients include solvents. Solvents may be subdivided into pharmaceutically acceptable and non-pharmaceutically acceptable solvents. In this context, it is to be understood that some pharmaceutically acceptable solvents include water for injection (WFI), which may be pH adjusted and/or buffered to a preselected pH or pH range, e.g. from about 2 to about 8, more specifically from about 4.0 to about 7.5, and more particularly from about 4.9 to about 7.2.

Pharmaceutically acceptable solvents may further comprise one or more pharmaceutically acceptable acids, bases, salts or other compounds, such as carriers, excipients, etc. Pharmaceutically acceptable acids include HCl, H₂SO₄H₃PO₄, benzoic acid, etc. Pharmaceutically acceptable bases include NaOH, KOH, NaHCO₃, etc. Pharmaceutically acceptable salts include NaCl, NaBr, KCl, etc. Acids and bases may be added in appropriate proportions to buffer a pharmaceutically acceptable solution at a particular, pre-selected pH, especially a pH in the range of about 2-8, more especially in the range of about 5.0 to about 7.2.

### Pharmaceutical Compositions

The invention provides a pharmaceutical composition comprising a therapeutically effective amount of an extract of Gleditsia sinensis Lam, wherein the therapeutically effective amount is effective to treat estrogen receptor (ER) negative breast cancer. In some embodiments described herein, the pharmaceutical composition is in the form of a medicament for treatment of estrogen receptor (ER) negative breast cancer comprising a therapeutically effective amount of an extract of Gleditsia sinensis Lam. Other embodiments described herein provide a pharmaceutical composition comprising a therapeutically effective amount of an extract of Gleditsia sinensis Lam, wherein the therapeutically effective amount is effective to treat a cancer that does not express an estrogen receptor (ER). The pharmaceutical composition is in the form of a medicament for treatment of a cancer that does not express an estrogen receptor (ER). A therapeutically effective amount of an extract of Gleditsia sinensis Lam includes an amount that provides relief from at least one symptom of the cancer, that reduces the size and/or rate of proliferation of the cancer. In some embodiments, the pharmaceutical composition or medicament further comprises one or more excipients. In some embodiments, the pharmaceutical composition of medicament consists essentially of one or more excipients and the extract of Gleditsia sinensis Lam. In some embodiments, the pharmaceutical composition or medicament consists of one or more excipients and the extract of Gleditsia sinensis Lam. In some embodiments, the pharmaceutical composition or medicament comprises, consists essentially of, or consists of one or more excipients for oral administration and the extract of Gleditsia sinensis Lam. In some embodiments, the pharmaceutical composition also includes an amount of an inhibitor or antagonist of the protein p38. In some embodiments the amount of p38 and the amount of the extract of Gleditsia sinensis Lam together are synergistic in the treatment of a cancer. In some embodiments the amount of p38 and the amount of the extract of Gleditsia sinensis Lam together are synergistic in the treatment of ER negative breast cancer. In some embodiments the amount of p38 and the amount of the extract of Gleditsia sinensis Lam together are synergistic in the treatment of PR negative breast cancer. In some embodiments the amount of p38 and the amount of the extract of Gleditsia sinensis Lam together are synergistic Her2/neu negative breast cancer. In some embodiments the amount of p38 and the amount of the extract of Gleditsia sinensis Lam together are synergistic against triple negative (ER, PR, and Her2/neu negative breast cancer).

In some embodiments, the therapeutically effective amount of the extract of Gleditsia sinensis Lam employed in the pharmaceutical composition or medicament is about 0.001 to about 100 grams dry weight of the extract per day. In some embodiments, the therapeutically effective amount of the extract of Gleditsia sinensis Lam is about 0.001 to about 10 grams dry weight, about 0.01 to about 10 grams dry weight, about 0.1 to about 10 grams dry weight, or about 1 to about 10 grams dry weight of the extract per day. Specific amounts of extract of Gleditsia sinensis Lam that may be administered in a 24 hour timeframe include about 50 mg dry weight, about 100 mg dry weight, about 150 mg dry weight, about 250 mg dry weight, about 300 mg dry weight, about 400 mg dry weight, about 500 mg dry weight, about 600 mg dry weight, about 700 mg dry weight, about 800 mg dry weight, about 900 mg dry weight, about 1 grams dry weight, about 2 grams dry weight, about 3 grams dry weight, about 4 grams dry weight, about 5 grams dry weight, about 6 grams dry weight, about 7 grams dry weight, about 8 grams dry weight, about 9 grams dry weight or about 10 grams dry weight.

In some embodiments, the therapeutically effective amount of the extract of Gleditsia sinensis Lam is about 1-100 grams dry weight of the extract per day. In some embodiments, the therapeutically effective amount is about 10 to about 100 grams dry weight, about 20 to about 100 grams dry weight, about 30 to about 100 grams dry weight, about 10 to about 80 grams dry weight, about 20 to about 80 grams dry weight, about 30 to about 80 grams dry weight, about 10 to about 60 grams dry weight, about 20 to about 60 grams dry weight, about 30 to about 60 grams dry weight, about 10 to about 50 grams dry weight, or about 20 to about 50 grams dry weight of the extract of Gleditsia sinensis Lam per day. Specific amounts of extract of Gleditsia sinensis Lam that may be administered in a 24 hour timeframe include about 10 grams dry weight, about 15 grams dry weight, about 20 grams dry weight, about 25 grams dry weight, about 30 grams dry weight, about 35 grams dry weight, about 40 grams dry weight, about 45 grams dry weight, about 50 grams dry weight, about 55 grams dry weight, about 60 rams dry weight, about 65 grams dry weight, about 70 grams dry weight, about 75 grams dry weight, about 80 grams dry weight, about 85 grams dry weight, about 90 grams dry weight, about 95 grams dry weight or about 100 grams dry weight.

The pharmaceutical composition or medicament may contain, in addition to the extract of Gleditsia sinensis Lam, one or more additional excipients, depending on the form of the pharmaceutical composition or medicament. Some suitable forms for administration to a patient include oral and parenteral dosage forms. Oral forms include liquid and solid dosage forms. Parenteral dosage forms are generally liquid. Suitable oral liquids generally contain water or other diluent and one or more additional excipients, such as one or more sweeteners, flavorings and/or taste-masking agents. Solid dosage forms include tablets and capsules, as well as powders and tablets that may be combined with water or other pharmaceutically acceptable diluent. Tablets generally contain one or more binders, and may also contain one or more dry solid diluents, dispersants, disintegrants, glidants, coatings, etc. Capsules may contain, in addition to the capsule shell itself, additional excipients, such as dispersants, disintegrants, etc. Powders for dissolution may contain, in addition to the dry extract of Gleditsia sinensis Lam, one or more flavorings, sweeteners and/or taste-masking agents. Alternatively, powders for dissolution can be packaged in a kit, with the dry extract of Gleditsia sinensis Lam (alone or in admixture with one or more excipients) in one container (e.g. a first pouch) and one or more excipients in a second container (e.g. a second pouch). Suitable containers for the dry extract of Gleditsia sinensis Lam and excipients may be air proof, water proof, light blocking, or combinations thereof.

In some embodiments, the extract of Gleditsia sinensis Lam is in an oral dosage form. In some embodiments, the oral dosage form is an elixir, a powder, one or more tablets, or one or more capsules. In some embodiments, the oral dosage is a concentrated oral elixir, optionally in admixture with one or more excipients, such as flavorings, sweeteners, and/or taste-masking agents. In some specific embodiments, a unit dose is a daily dose or a divided daily dose. A daily dose may be in a single dosage unit or may be divided between 2, 3, 4 or more dosage units. In some currently preferred embodiments, the daily dose may be given as a single dose, once per day (q.d.) and the daily dose is contained in a single dosage unit. In some other currently preferred embodiments, the daily dose is given as two separate doses (b.i.d.) and the daily dose is contained in separate dosage units, which may conveniently be connected to one another, sealed in a common container or otherwise associated with one another as to form an easily identifiable daily dosage unit. In a currently preferred embodiment, the daily dose is evenly divided between two separate dosage units, although in other embodiments the daily dose need not be evenly divided between separate dosage units.

The specific cancers that may be treated with the pharmaceutical compositions and medicaments according to the present invention include those in which an extract of Gleditsia sinensis Lam induces apoptosis. Cancers that have been found to be susceptible to inducement of apoptosis by extract of Gleditsia sinensis Lam include those cancers that do not express estrogen receptor, and especially those that do no express the estrogen receptor alpha (ERα) Thus, some cancers that may be treated with a therapeutically effective amount of extract of Gleditsia sinensis Lam include estrogen receptor negative breast cancer, progesterone negative breast cancer, Her2/neu negative breast cancer, breast cancer that is negative for two or all three of ER, PR and Her2/neu. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

Extracts of Gleditsia sinensis Lam may be prepared as above in either solution or dried form. In a solution form, an extract of Gleditsia sinensis Lam may be administered as a flavored or unflavored tea. Thus, excipients include, in some embodiments some flavoring, e.g. sweetening, which may be desirable to counteract the bitter flavor of the extract. Solutions can also be prepared from dried extract, in tea or elixir forms. Again, flavoring, such as sweetening may be desirable. Taste-masking may be employed to improve patient acceptance of the pharmaceutical composition. Sweeteners, include

A dried extract may be formulated as an orally-available form, such as in a capsule, tablet, caplet, etc. A capsule may be prepared by measuring a suitable amount of the dry extract into one or more gelatin capsule shells and assembling the capsule(s). Tablets and caplets may be prepared by combining the dry extract with one or more binders and optionally one or more disintegrants. Tablets, caplets, capsules, etc. may be coated, e.g. with an enteric coating, to prevent stomach upset.

The dried extract mentioned above can also be prepared in a powder form that is capable of being dissolved in water or other suitable solvent and administered to the patient. In some embodiments, this form is an oral form. In some specific examples, the powder may

Either a dried extract or a concentrated extract solution may be combined with one or more gelling agents and inserted into a gel capsule. Alternatively, a dried extract or concentrated extract solution may be combined with a gelling agent and optionally one or more flavoring agents for oral administration as an edible gel or a non-flavored variant may be administered as a rectal suppository gel or gel capsule.

A unit dose of extract is characterized by an equivalent amount of dried extract contained within the dosage form. For example, in some embodiments, a unit dosage may contain 1 mg to about 10 g of dried extract, or the equivalent thereof. In some embodiments, the unit dose will contain about 1 mg to about 10 mg, about 1 mg to about 100 mg, about 1 mg to about 1000 mg (1 g), about 1 mg to about 10000 mg (10 g) of dried extract, or the equivalent thereof. In some embodiments, the unit dose contains about 10 mg to about 100 mg, about 10 mg to about 1000 mg or about 10 mg to about 10000 mg of dried extract or the equivalent thereof. In some embodiments, the unit dose contains about 100 mg to about 5000, about 100 mg to about 2500 mg, about 100 mg to about 2000 mg, about 100 mg to about 1500 mg, about 100 to about 1000, about 100 to about 800 mg of dried extract, or the equivalent thereof. An equivalent of a dried extract of Gleditsia sinensis Lam is an amount of a dry, liquid, gel or other mixture of Gleditsia sinensis Lam containing the same amount of apoptotic active as a dried extract of Gleditsia sinensis Lam. Thus, 30 mL of a tea containing 0.090 mg/mL of dried extract of Gleditsia sinensis Lam is a unit dose equivalent to 15 mg of dried Gleditsia sinensis; and a tablet containing 100 mg each of dried extract of Gleditsia sinensis, a binder, a filler, a disintegrant is equivalent to 100 mg of dried extract neat. Other dosages, such as those in the 10-100 grams dry weight per day range, are also contemplated, as described in more detail herein.

In some embodiments, the pharmaceutical compositions contain a p38 MAP kinase inhibitor. In some embodiments, the p38 MAP kinase inhibitor is SB203580, SB202190, SB239063, LY479754, ARRY-797, ARRY-614, LP-590, PD169316, VX-702, or a pharmaceutically acceptable salt or combination thereof. The p38 MAP kinase inhibitors are compounds that inhibit the mitogenic MAP kinase, which is involved in inflammatory response and has been implicated in apoptosis, potentially as protecting cells from apoptosis.

### Methods of Treatment

The compositions comprising extracts of Gleditsia sinensis Lam as described herein possess selective Gleditsia sinensis Lam have apoptotic actively in estrogen receptor negative (ER-negative) cancer cells, such as ER-negative breast cancer and prostate cancer cells. Hence, it is expected that they will have activity in the treatment of various disease states that are characterized by hyperproliferation of cells, such as those caused by failure of normal apoptotic processes in an organism, organ, tissue or cell line. Among the disease states envisioned as being treatable with the compositions described herein is cancer, including, but not limited to bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer or uterine cancer. In some embodiments the composition described herein is administered to a patient who has been diagnosed with one or more cancers selected from among the solid tumors, such as breast, lung, colon, brain, prostate, stomach, pancreatic, ovarian, skin (melanoma), endocrine, uterine, testicular and bladder cancer.

In some embodiments, compositions comprising extracts of Gleditsia sinensis Lam described herein are effective to treat a benign proliferative disease, such as benign prostatic hypertrophy, psoriasis or restenosis (e.g. of an implanted stent).

In some embodiments, one or more compositions comprising extracts of Gleditsia sinensis Lam described herein may be combined with another agent that is useful for the treatment of abnormal cell growth, such as cancer, solid tumors, benign hyperproliferative disease, etc. Such additional agent may be selected from among the mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic agents, anti-hormones, and anti-androgens. Other additional agents include p38 MAP kinase inhibitors, such as SB203580, SB202190, SB239063, LY479754, ARRY-797, ARRY-614, LP-590, PD169316, VX-702, or a pharmaceutically acceptable salt or combination thereof.

An effective dose of a composition comprising an extract of Gleditsia sinensis Lam is an amount effective to produce a therapeutic effect in a patient as described herein. In some embodiments, the effective dose is an amount sufficient to induce apoptosis in one or more populations of hyperproliferative cells in the patient. In some embodiments, the effective dose is an amount sufficient to cause relief of one or more symptoms of hyperproliferative cellular disease, such as cancer, in the organism. In some embodiments, the effective dose is an amount sufficient to significantly slow the progression of hyperproliferative cellular disease, to cause partial or complete remission of said hyperproliferative cellular disease, to provide partial or complete prophylaxis against recurrence, spread or malignant growth of said hyperproliferative cellular disease. In some embodiments the dose may be critical to the success of the therapeutic regime. As the extracts of Gleditsia sinensis Lam are deemed to be largely non-toxic, the effective dose may be varied from about 1 mg to about 100 g per patient per day of dried extract, or the equivalent thereof in a solution or other pharmaceutically acceptable form, as discussed in more detail below. In some embodiments, the effective dose is about 1 mg to about 10 mg, about 1 mg to about 100 mg, about 1 mg to about 1000 mg (1 g), about 1 mg to about 10000 mg (10 g) per patient per day. In some embodiments, the effective dose is about 10 mg to about 100 mg, about 10 mg to about 1000 mg or about 10 mg to about 10000 mg per patient per day. In some embodiments, the effective dose is about 100 mg to about 5000, about 100 mg to about 2500 mg, about 100 mg to about 2000 mg, about 100 mg to about 1500 mg, about 100 to about 1000, about 100 to about 800 mg per patient per day. In some embodiments, the daily dose is in the range of about 10 grams dry weight to about 100 grams dry weight of Gleditsia sinensis Lam per day, as described in more detail herein.

Thus, in some embodiments of the invention described herein provide a method of treating a patient having estrogen receptor (ER) negative breast cancer, comprising administering a therapeutically effective amount of an extract of Gleditsia sinensis Lam effective to the patient. Addition embodiments described herein provide a method of treating a patient having cancer that does not express an estrogen receptor (ER), comprising administering a therapeutically effective amount of an extract of Gleditsia sinensis Lam effective to the patient. In some embodiments, the therapeutically effective amount of the extract of Gleditsia sinensis Lam is about 0.001 to about 100 grams dry weight of the extract per day. In some embodiments, the therapeutically effective amount of the extract of Gleditsia sinensis Lam is about 0.001 to about 10 grams dry weight of the extract per day. In some embodiments, the therapeutically effective amount of the extract of Gleditsia sinensis Lam is about 1-100 grams dry weight of the extract per day. In some embodiments, the ER negative breast cancer is estrogen receptor alpha (ERα) negative. In some embodiments, the ER negative breast cancer is also negative for one or both of progesterone receptor (PR) and/or Her2/neu. In some embodiments, the ER negative breast cancer is triple negative breast cancer. In some embodiments, the ER negative breast cancer is metastatic. In some embodiments, the extract of Gleditsia sinensis Lam is in an oral dosage form. In some embodiments, the oral dosage form is an elixir, a powder, one or more tablets, or one or more capsules. In some embodiments, the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

In some embodiments, treatment days may be altered with non-treatment days. For example, treatment may be commenced on day 1 with an effective dose as described above, with administration of the effective dose repeated on days 3, 5, 7 (or 8), 9, 11, 13, etc. Treatment may be administered once a day for a full week, followed by a week off treatment, followed by at least one additional week on treatment. Treatment with the extract of Gleditsia sinensis Lam may also be alternated with another anti-cancer treatment, or may be combined with another anti-cancer treatment to take advantage of the combined effects of the cancer treatments.

Additional cancer treatments can include, but are not limited to, surgical excision of all or part of a solid tumor, radiation treatment, adjunctive chemotherapy, anti-inflammatory drugs, analgesic drugs, etc.

### Previous Therapies

In some embodiments, the invention provides for administering a therapeutically effective amount of an extract of Gleditsia sinensis Lam to a patient for the treatment of cancer, particularly cancer that has failed to respond to one or more previous therapies. Various therapies for the treatment of cancer are known, and may be considered as antecedents to the use described herein. For example, breast cancer patients often undergo surgical removal of the cancerous lesion, e.g. lumpectomy (also known as wide local excision), or mastectomy. In some cases the lymph nodes are also removed (radical mastectomy). In some cases, the patient may undergo radiation therapy, either instead of, or more commonly as an adjunct to surgical removal of the lesion. In some cases, the patient may undergo chemotherapy, as an alternative or adjunct to surgery and/or radiation treatment. It is not uncommon for a patient to undergo one or more of the foregoing treatments only to find at a later date that the tumor has spread or metastasized to neighboring or even distal tissue. It is considered an aspect of the invention that a pharmaceutical composition comprising a therapeutically effective amount of an extract of Gleditsia sinensis Lam be administered to a patient who has undergone previous surgical removal of a cancerous lesion, prophylactic removal or partial removal of the breasts, radiation treatment and/or chemotherapy. In some embodiments, the treated cancer has proven refractory to the previous surgery, radiation treatment and/or chemotherapy.

Previously used chemotherapies include chemotherapy with one or more chemotherapeutic agents. Particular chemotherapeutic agents that are available to treat breast cancer, including cytotoxic drugs such as doxorubicin, cyclophosphamide, methotrexate, paclitaxel (Taxol®, Abraxane®), docetaxel, thiotepa, mitoxantrone, vincristine, tamoxifen, megestrol acetate, aminoglutethimide, fluoxymesterone, leuprolide, goserelin, prednisone, or combinations thereof.

Particular chemotherapeutic agents that are available to treat ovarian cancer include cyclophosphamide, etoposide, altretamine, tamoxifen, and combinations thereof.

Particular chemotherapeutic agents that are available to treat cervical cancer include cisplatin, carboplatin, hydroxyurea, irinotecan, bleomycin, vincristine, mitomycin, ifosfamide, fluorouracil, etoposide, methotrexate, and combinations thereof.

Particular chemotherapeutic agents that are available to treat prostate cancer include doxorubicin, estramustine, etoposide, mitoxantrone, vinblastine, paclitaxel, docetaxel, carboplatin, and prednisone.

Particular chemotherapeutic agents that are available to treat pancreatic cancer include 5-fluorouracil (5-FU), mitomycin, ifosfamide, doxorubicin, streptozocin, chlorozotocin, and combinations thereof.

Particular chemotherapeutic agents that are available also include the VGFR and EGFR inhibitors, such as gefitinib, erlotinib, imatinib, and combinations thereof.

### DESCRIPTION OF SPECIFIC TERMS

As used herein, the term “method” refers to manners, means techniques and procedures for accomplishing a given task including, but not limited to, those manners, means techniques and procedures either known to, or readily developed from known manners, means, techniques and procedures by, practitioners of the chemical, pharmacological, biological, biochemical, medical, and homeopathic arts.

As used herein, “inhibiting the activity” refers to slowing, preferably stopping, the growth and/or proliferation of cancerous cells, both in-place, i.e., growth and proliferation at the initial site of tumor formation, and proliferation by metastasis. Inhibiting the activity also encompasses, in fact it is the most preferred embodiment of this invention, killing cancerous cells.

As used herein, the term “cancer” refers to various types of malignant neoplasms, most of which can invade surrounding tissues, and may metastasize to different sites, as defined by Stedman's Medical Dictionary 25^{th} edition (Hensyl ed. 1990). Examples of cancers which may be treated by the present invention include, but are not limited to, brain, ovarian, colon, prostate, kidney, bladder, breast, lung, oral and skin cancers. In a presently preferred embodiment of this invention the cancer being treated is breast or ovarian cancer.

As used herein, the term “contacting” in the context of contacting a solid tumor cancer cell with an extract of this invention bringing an extract of this invention and a target cancer cell together in such a manner that the extract can affect the activity of the cell either directly or indirectly. As used herein, contacting refers to procedures conducted in vitro, i.e. cancerous cells which are the object of this invention are studied, outside a patient. Cells existing outside the patient can be maintained or grown in cell culture dishes. For cells outside the organism, multiple methods exist, and are well-known to those skilled in the art, to contact extract of this invention, with or without employment of various well-known transmembrane carrier techniques and direct cell microinjection

The term “in vivo” refers to contacting or treatment within a living organism, such as a living human or other mammal, such as a mouse or rat.

As used herein, an “extract” refers to the residue of soluble solids obtained, either in dry or solubilized form, after Gleditsia sinensis Lam, or selected part thereof has been subjected to an extraction process, preferably in water, alcohol or combination thereof.

As used herein, “BN107” refers to an extract of Gleditsia sinensis Lam.

As used herein, the terms “treat”, “treating” and “treatment” refer to a method of alleviating or abrogating a solid tumor cancer and/or its attendant symptoms. In particular, the terms simply mean that the life expectancy of an individual affected with a cancer will be increased or that one or more symptoms of the disease will be reduced.

As used herein, “administer”, “administering” or “administration” refers to the delivery of an extract or extracts of this invention or of a pharmaceutical composition containing an extract or extracts of this invention to a patient in a manner suitable for the treatment of particular cancer being addressed. The term includes self-administration and administration by a health care professional or other care provider.

As used herein, the term “mammal” refers to any mammal that is affected by a cancer, whether that cancer is autologous (i.e. arises naturally in the mammal) or is of xenogenous (i.e. xenogenic) origin. The term “mammal” includes humans, as well as murine, canine, feline, equine, bovine, ovine, porcine and other mammalian species.

A “patient” refers to any higher organism that is susceptible to solid tumor cancers. Examples of such higher organisms include, without limitation, mice, rats, rabbits, dogs, cats, horses, cows, pigs, sheep, fish and reptiles. In particular examples, “patient” refers to a human being. In particular embodiments, the patient is a human suffering from cancer, such as breast cancer or other cancer described herein. In some embodiments, the cancer is a metastatic cancer, such as metastatic breast cancer or other metastatic cancer described herein. In some embodiments, the patient is treatment-naïve; in some preferred embodiments, the patient has previously undergone treatment for cancer. In some embodiments, the patient is currently undergoing other treatment for cancer. In some embodiments, the patient has previously been treated with one or more cancer therapies, but has failed to respond to therapy. In some embodiments, the patient has been previously treated with one, two, three, four or more, particularly 1-4, previous therapies but has failed to respond to those therapeutic approaches. Thus, a preferred subclass of “patient” according to this invention is a patient suffering from metastatic breast cancer who has previously been treated with, but failed to respond to, one to four previous therapies for the breast cancer.

As used herein, the term “therapeutically effective amount” refers to an amount of extract of Gleditsia sinensis Lam that is effective to treat at least one symptom of cancer in a patient. In particular embodiments, such an amount of an extract has at least one effect from the following list: (1) reducing the size of the tumor; (2) inhibiting (that is, slowing to some extent, preferably stopping) tumor metastasis; (3) inhibiting to some extent (that is slowing to some extent, preferably stopping) tumor growth; and/or; (4) relieving to some extent (or preferably eliminating) one or more symptoms associated with cancer; (5) stabilizing the growth of the tumor, (6) extending the time to disease progression; and/or (7) improving overall survival.

As used herein, a “pharmaceutical composition” refers to a mixture of one or more of the extracts described herein with other chemical components, such as physiologically acceptable carriers and excipients. The purpose of a pharmacological composition is to facilitate administration of an extract or extracts of this invention to patient.

As used herein, the term “pharmaceutically acceptable” means that the referenced agent or excipient is generally regarded as acceptable for use in a pharmaceutical composition.

As used herein, a “physiologically acceptable carrier” refers to a carrier or diluent that does not cause significant irritation to an organism and does not abrogate the biological activity and properties of the administered composition.

As used herein, an “excipient” refers to an inert substance added to a pharmaceutical composition to further facilitate administration of an extract of this invention. Thus, the term “excipient” specifically excludes other active ingredients, such as, in particular, other chemotherapeutic ingredients, including, but not limited to, ingredients derived from plant species other than Gleditsia sinensis Lam.

As used herein, the terms “comprising”, “comprises”, “comprise” and grammatical variants thereof are inclusive or open-ended and do not exclude additional, unrecited elements or method steps. The terms “include”, “includes”, “contain”, “contains”, “containing” and grammatical variants thereof are likewise inclusive.

As used herein, the phrase “consisting of” excludes any element, step, or ingredient not specified in the following portion of the sentence.

As used herein, the phrase “consisting essentially of” limits the scope of the following part of the sentence to the specified materials or steps and those that do not materially affect the basic and novel characteristic(s) of the claimed invention. In the present case, an active pharmaceutical ingredient known to have anti-cancer activity would be considered a material that would materially affect the basic and novel characteristics of the claimed invention, whereas an analgesic or antiinflammatory would not.

As used herein, the term “grams dry weight per day” (also “gm dry weight”) means, in reference to an extract of Gleditsia sinensis Lam, the dry weight, in grams, of the residue after a quantity of Fructa Gleditsia sinensis Lam has been extracted and the extraction medium has been removed, e.g. by evaporation or freeze drying.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the selectively apoptotic plant extract of the invention to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the herein-described plant extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a plant extract of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a plant extract of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

### EXAMPLES

The invention may be more fully appreciated with reference to the following illustrative and non-limiting examples.

### Example 1

### In Vitro Studies

Extracts of Gleditsia sinensis Lam fruit appear to exert their growth inhibition properties on breast cancer cells via the mitochondrial apoptotic pathway. Absence of estrogen receptor (ER) in the cells correlates with sensitivity to extract of Gleditsia sinensis Lam. Introduction of ERα expression into a breast cancer line results in protection from the pro-apoptotic effect of Gleditsia sinensis Lam. Transcriptomic analysis comparing sensitive (ER⁻) and insensitive (ER⁺) lines treated with of Gleditsia sinensis Lam extract revealed distinct patterns of gene expression that might be responsible for the differential sensitivity.

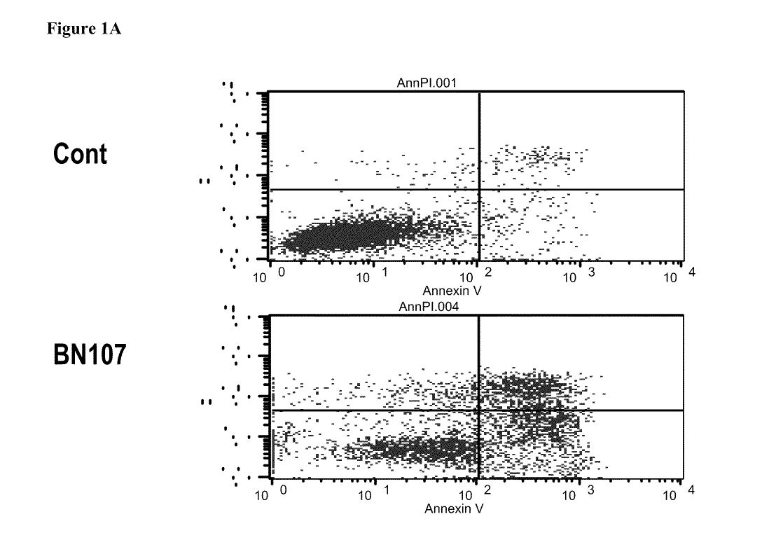

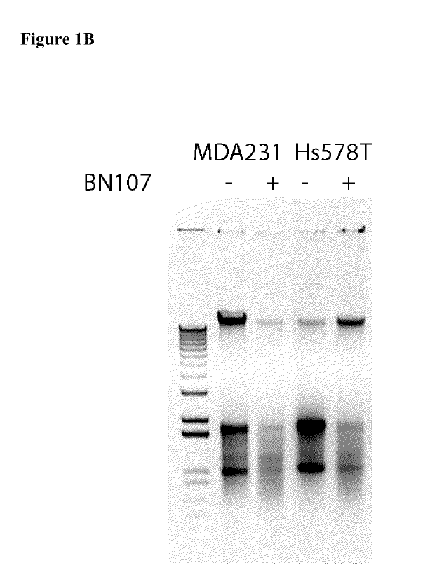

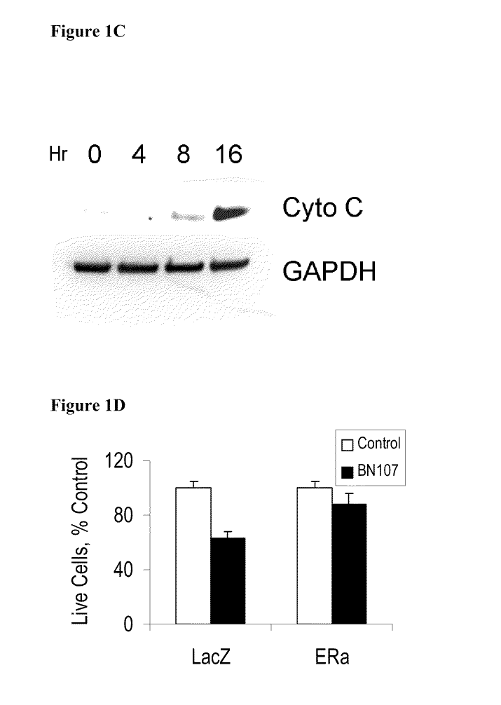

Plant extracts of Gleditsia sinensis Lam selectively induce apoptosis in cancerous cells. Tumor and non-transformed cell lines and cells were treated with a solution comprising 0.090 mg/mL (90 μg/mL) of dried extract of Gleditsia sinensis Lam fruit. The solution containing 0.090 mg/mL of dried extract of Gleditsia sinensis Lam fruit is also referred to herein as BN107. As can be seen, the graph in FIG. 1A-1D show the percentage of cells that bound Annexin V after 24 hours of treatment.

ERα plays a role in BN107-induced apoptosis. In Table 1-1 below are the results of experiments in which cells were treated with BN107 and harvested after 24 hours for analysis of Annexin V/PI binding. Three independent experiments were conducted for each data point.

TABLE 1-1
Table 1: Cells were treated with BN107 and harvested after
24 hours for analysis of Annexin V/PI binding. The summary
shown is a result of 3 independent experiments.

| | Annexin V PI | | | | |
|---|---|---|---|---|---|
| | staining | Her2 | ERα | p53 | C-Myc |
| SKBr3 | ++ | + | − | M175 | Low |
| Hs578T | ++++ | + | − | M | High |
| MDA-MB-468 | ++ | − | − | M273 | Med |
| MDA-MB-231 | ++++ | − | − | M280 | Med |
| MDA-MB-453 | ++++ | + | − | WT | High |
| MCF10A | +++ | − | − | WT | Low |
| IMR90 | ++++ | − | − | WT | |
| MDA-MB-361 | −/+ | + | + | WT | High |
| BT474 | −/+ | + | + | M285 | High |
| MCF7 | − | − | + | WT | Med |

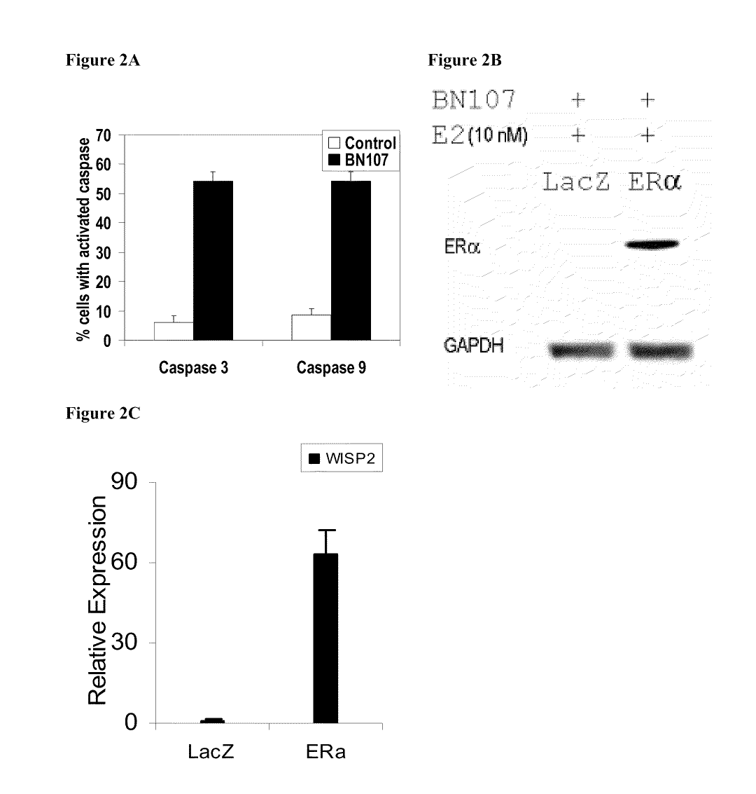

Interestingly, ERα expression rescues cells from BN107-induced apoptosis, as shown in FIG. 2. In particular, FIG. 2A shows the results of an experiment in which MDA-MB231 cells infected with LacZ or Era virus were treated with BN107 in the presence of estrogen (10 mM) and analyzed with Annexin/PI binding. BN107 caused cell death in just under half of the LacZ cells, whereas relatively little cell death was seen in the Era cells. As shown in FIG. 2B, LacZ cells were ERα negative (ERα⁻), whereas the Era cells were positive for ERα protein (ER⁺). As shown in FIG. 2C, expression of WISP2, a downstream target of ERα, is nearly obliterated in the LacZ cells, whereas in the Era cells produced significant amounts of WISP2. Table 1-2, below, summarizes Ingenuity Pathway Analysis (IPA) of microarray data generated using BN107-sensitive (Hs578T) and insensitive (MCF7) cells treated with BN107.

TABLE 1-2
Table 2: Cellular/signaling pathways induced by BN107 treatment,
based on Ingenuity Pathway Analysis (IPA) of microarray
data generated using BN107 sensitive (Hs578T) and insensitive
(MCF7) cells treated with BN107.

| Hs578T (ER−) | MCF7 (ER+) |
|---|---|
| Apoptosis | Ah receptor signaling |
| Cell cycle | IGF1 receptor signaling |
| Oxidative response | MAPK signaling |
| MAPK signaling | Cell growth |
| Acute response | Acute response |

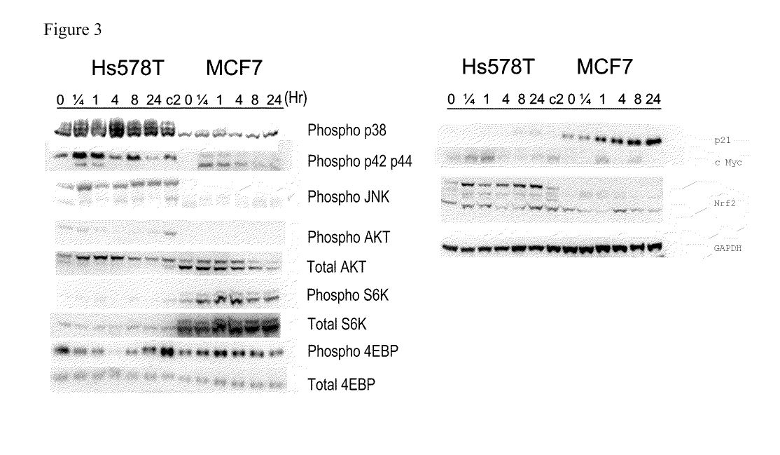

FIG. 3 shows the results of protein expression analysis on Hs578T and MCF7 cells treated with BN107. Hs578T and MCF7 cells were treated with BN107 and harvested at the indicated time points.

The in vitro experiments demonstrate that the Mitochondrial-mediated apoptosis appears to be the major cellular pathways mediating the growth inhibitory effect of BN107. The presence of functional estrogen receptor renders breast cancer cells less sensitive to BN107 induced apoptosis. Cells sensitive to BN107 respond by increasing expression of pro-death molecules and inhibiting pro-survival/growth pathways; while resistant cells respond by committing cells in cell cycle arrest and/or increasing activities of proteins involving in cell growth. Thus, it is concluded that BN107, and by extension extracts of the fruit of Gleditsia sinensis Lam in general, possess selective apoptotic activity. It is thus expected that pharmaceutical compositions comprising extracts of the fruit of Gleditsia sinensis Lam will have apoptotic activity in multicellular organisms, especially in tissues that do not express ERα, such as ERα-negative breast cancer, prostate cancer, etc.

### Example 2

### The Selective Pro-Apoptotic Effect of BN107 and Oleanolic Acid on Estrogen Receptor Negative Breast Cancer Cells is Mediated by Disruption of mTORC1/mTORC2 Survival Signaling on Lipid Rafts

Hormonal, targeted or chemotherapeutic strategies largely depend on the expression of their cognate receptors and are often accompanied by intolerable toxicities. Effective and less toxic therapies against the estrogen receptor negative (ER−) breast cancer are urgently needed. This example explores the potential mechanisms mediating the selective pro-apoptotic effect induced BN107 and its principle saponin, oleanolic acid (OA), on ER− breast cancer cells.

A panel of breast cancer cell lines was examined and the most significant cytotoxic effect was observed in the ER− breast lines. Apoptosis appeared to be the major cellular pathway mediating the cytotoxicity of BN107. The sensitivity to BN107 was greatly reduced when ERα expression was introduced in MDA-MB-231, confirming the protective role of ERα on BN107-induced apoptosis. BN107, an extract rich in OA derivatives, caused rapid alterations in cholesterol homeostasis, presumably by binding to cholesterol which interfered with plasma membrane lipid rafts (LR) and signaling mediated by LR. BN107 or OA treatment in ER− cells resulted in rapid and specific redistribution or degradation/displacement/inhibition of important survival signaling complexes that are associated with LR, namely mTORC1, mTORC2 and Akt. Co-administration of BN107 or OA with cholesterol specifically abolished the pro-apoptotic effect and restored the disrupted survival signaling. This demonstrates concomitant inhibition of mTORC1/mTORC2/Akt activities by modulating the levels of protein constituents present in these signaling complexes.

Despite advances in treatment options have made a favorable impact on survival, current regimens lead to toxic side effects and are mostly ineffective against estrogen receptor negative (ER−) metastatic breast cancer. Currently, patients with ER−/progesterone receptor negative (PR−)/HER2 negative (Her2−) tumors still present a therapeutic challenge for the oncologists. Therefore, novel and effective therapies with minimal toxicities are urgently needed for this patient population.

The anti-cancer effect of the fruit of Gleditsia sinensis Lam or Gleditsia saponins that were isolated from it have been attributed to induction of cytotoxicity which might be related to their abilities to induce reactive oxygen species (ROS), inhibit telomerase, COX2 expression, VEGF secretion and proteasome activity. BN107 is an aqueous extract of the G. sinensis that has been shown to exhibit anti-proliferative activity on a panel of human breast cancer lines. The extract of G. sinensis is enriched with triterpenoidal saponins that possess similar base structure as oleanolic acid (OA). These saponins have been shown to exhibit differential cytotoxicities against tumor cells which depend greatly on the presence and position of the oligosaccarides chains and the monoterpene units. In addition, OA, and its synthetic derivatives have been shown to induce strong anti-tumor activity, in a wide variety of tumor cells in culture and in animal models.

The physiological activity of triterpenoidal saponins is usually associated with their ability to complex plasma membrane cholesterol. It is now well established that cholesterol is important for the functions of lipid rafts (LRs), a specialized platform within plasma membrane, and that agents which bind and/or extract cholesterol from the rafts alter the localization and the functions of the raft-associated proteins. LRs are sites where cell surface receptors and signaling molecules are concentrated and which spatially organize signal transduction at the cell surface. LRs have been implicated in processes as diverse as viral infection, endocytosis, cholesterol trafficking, and cell growth and survival. It has been shown that some proteins selectively partition into the LRs. These include glycosylphosphatidylinositol-anchored proteins, myristoylated or palmitoylated proteins (such as Akt, flotillin), doubly acylated proteins (such as Src-family kinases), phospholipid bound proteins (such as annexins), and cholesterol-bound transmembrane proteins (such as caveolins). Other examples of proteins/protein complexes involved in growth and survival have also been shown to partition into LRs. Specifically, approximately 60% of the receptor tyrosine kinases (i.e. EGFR, PDGFR) are localized to LRs. In addition, LRs have also been shown to provide a “platform” for proper assembly of functional protein complexes. For example, mTOR activities have been shown to depend on the presence of the complex components residing on LRs.

Akt/mammalian target of rapamycin (mTOR) pathway is the prototypic survival pathway that is aberrantly activated in many types of cancer. This pathway is central in the transmission of growth regulatory signals and survival originating from cell surface receptors. Growth factors and cytokines activate Akt via PI3 kinase (PI3K), which phosphorylates phosphatidylinositol-4,5-bisphosphate [PI(4,5)P2] to generate [PI(1,4,5)P3] that binds to the PH domain of Akt and phosphoinositide-dependent protein kinase 1 (PDK1), recruiting them to the plasma membrane. Once in the membrane, Akt is phosphorylated by PDK1 at Thr308 and by mTORC2 at Ser 473. When Akt is fully activated, signaling through Akt can be propagated to a diverse array of substrates, including mTORC1, a key regulator of protein translation. Akt activation also regulates anti-apoptotic genes such as Bcl-xL and FLIP.

mTOR is a serine/threonine kinase that regulates a variety of cellular activities that are sensitive to environmental stress. Although activating mutations in mTOR itself have not been identified, de-regulation of upstream components that regulate mTOR activities is prevalent in cancers. Recently, a component of mTOR protein complex, RICTOR, has been shown to overexpress in hepatocellular carcinoma and glioma. The prototypic mechanism of mTOR regulation in cells is through activation of the PI3K/Akt pathway, but mTOR receives input from multiple signaling pathways. In mammalian cells two mTOR/FRAP1-containing complexes have been identified, mTOR complex1 (mTORC1) and mTOR complex 2 (mTORC2). mTORC1 is comprised of mTOR/FRAP1, RAPTOR, and mLST8. Whereas the function of mLST8 is not fully clarified, RAPTOR functions as a scaffold for recruiting mTORC1 substrates, such as the p70S6K (ribosomal p70S6 kinase) and 4E-BP (eukaryotic initiation factor 4E binding protein), both regulators of protein translation. mTORC2 contains mTOR/FRAP1, RICTOR, mLST8, sin 1, and the recently identified protor. RICTOR and sin 1 appear to stabilize each other through binding, building the structural foundation for mTORC2. Activated mTORC2 regulates the actin skeleton and phosphorylates Akt at Ser473, which in conjunction with PDK1-mediated phosphorylation drives full activation of Akt. It has been proposed that mTOR/FRAP1 polypeptide and other complex components of mTORC1 and mTORC2 reside on the LRs and mTOR/FRAP1 polypeptide is shared between mTORC1 and mTORC2 complexes. Therefore, when mTORC2 complex is disrupted, which frees up mTOR/FRAP1 polypeptide, mTORC1 activity reciprocally increases.

Conversely, when mTORC2 components are recruited to LRs, Akt is activated in the raft by mTORC2 kinase activity.

Given that mTOR is a nodal regulator of cellular survival, enormous efforts have been put in to develop molecules against mTOR activity in cancer therapy. mTOR inhibitors, rapamycin and derivatives (rapalogs) have been developed to target mTORC1 complex; while mTORC2 is relatively insensitive to rapamycin, albeit recent evidence shows that prolonged incubation with rapamycin also decreases the activities of mTORC2. mTORC1 blockade is expected to lead to significant anti-tumor effects in tumor cells in which the PI3K pathway is constitutively active. Indeed, some rapalogs have shown promising anti-tumor activities in Akt-dependent prostate cancer, Neu/Erb2 dependent breast cancer or PTEN deficient tumor models. In addition, some rapalogs have recently provided significant activities in the treatment of metastatic real cell carcinoma. Activities against other solid tumors, including breast, are not as impressive. The molecular mechanisms responsible for these differences in sensitivity have not yet been clearly underlined. Evidence exists showing that mTORC1 inhibition can lead to pathway reactivation: abrogation of the negative-feedback loop which is normally initiated by the direct mTORC1 substrate p70 S6 kinase that can lead to strong PI3K-Akt reactivation. Moreover, rapalogs cannot inhibit mTORC2 efficiently, which is one of the two upstream Akt kinases. Altogether, this would suggest that pathway activation and reactivation could be avoided by agents that lead to concomitant Akt and mTOR inhibition (that would target both mTORC1 and mTORC2).

The experiments below elucidate the mechanism of action of BN107 and OA on inducing apoptosis selectively in ER− breast cancer cells by disrupting the survival signaling mediated by LRs. In particular, BN107 or OA selectively disrupts both the mTORC1 and mTORC2 complexes residing on the LRs; thereby leading to displacement/downregulation of mTOR complexes components and their activities. Inhibition of mTORC2 activities further inactivates Akt signaling selectively in the ER− breast cancer cells. To the best of our knowledge, these findings provide the first evidence that oleanolic acid as a single agent to inhibit Akt and mTOR (mTORC1 and mTORC2) activities concomitantly.

Result

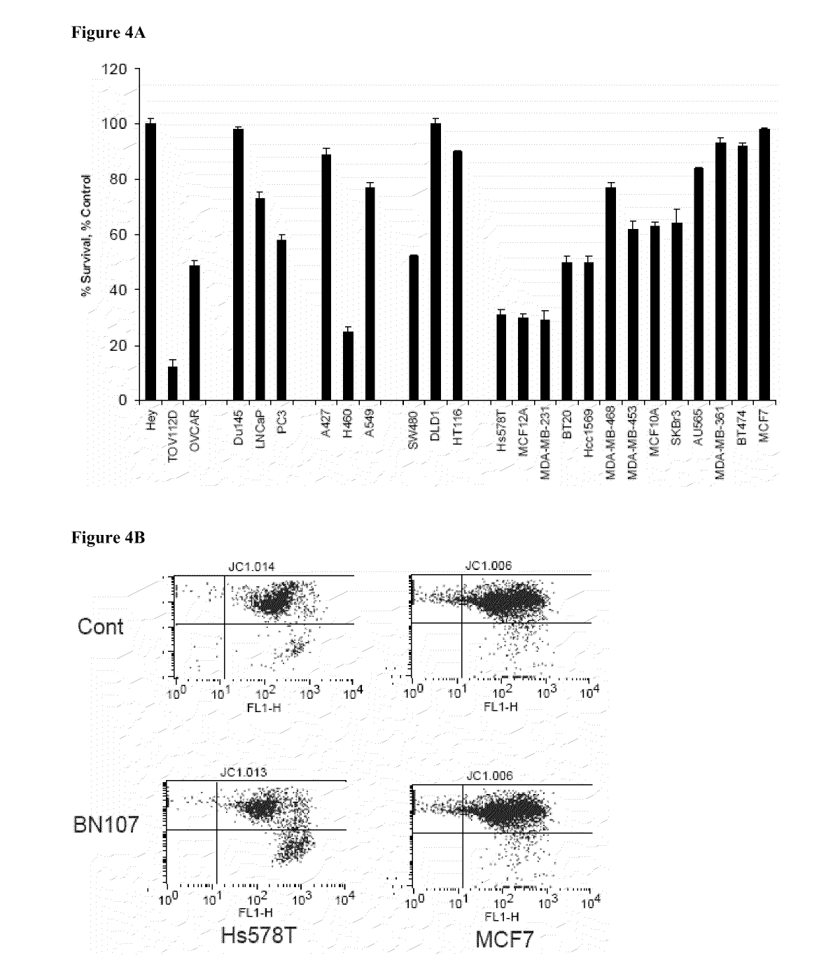

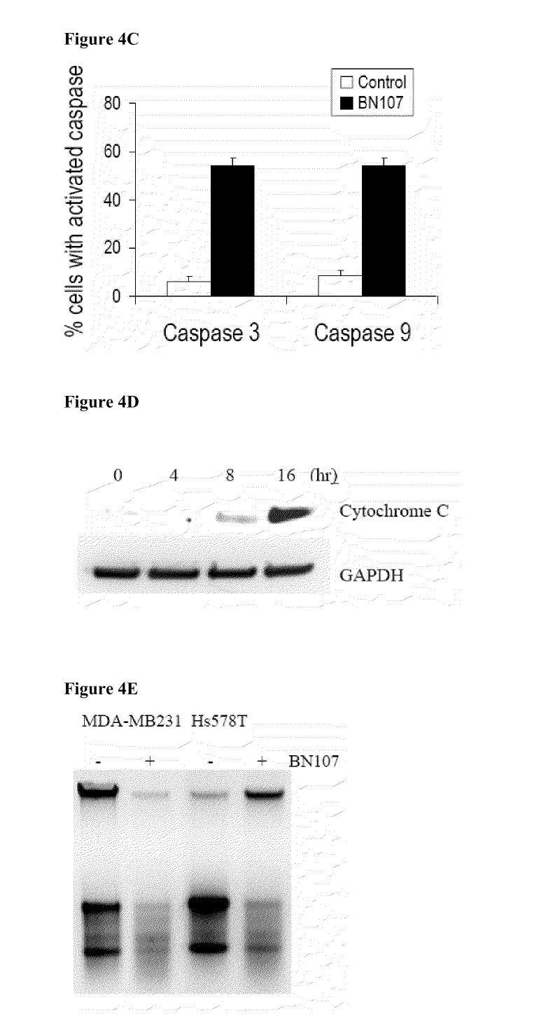

BN107 induced apoptosis selectively in ER− breast cancer cells and introducing expression of ERα protected these cells against BN107 Prior studies have shown that ethanolic extract of G. sinensis is cytotoxic to a number tumor cell lines by inducing cytotoxicity. A wider panel of solid-tumor cell lines, derived from various origins, is analyzed herein. Sub-confluent cultures were treated with 70 μg/ml of BN107 for 18 hours and cell death were analyzed by annexinV/PI binding followed by flow cytometry. FIG. 4A shows the percentage of survival cells (annexin V−, PI−) at a dose that killed ˜50% of the MDA-MB-231 cells, previously shown to be sensitive to BN107. It is evident that BN107 induced cell death in tumor lines derived from various origins to different extent. Specifically in breast lines, the cells displayed a wide range of sensitivity towards BN107. These experiments sought to determine if there was correlative relationship between genotypic characteristics of the cells versus sensitivity. As shown in Table 2-1, it appeared that cells lacking ER expression were highly sensitive to BN107; while cells containing functional ER were relatively insensitive to BN107 at this dose. The death induced by BN107 in ER− lines was apoptotic in nature that was primarily mediated by the mitochondrial pathway, as evident from Annexin V binding, dissipation of mitochondrial potential, cytosolic release of cytochrome C, activation of caspases, and DNA fragmentation.

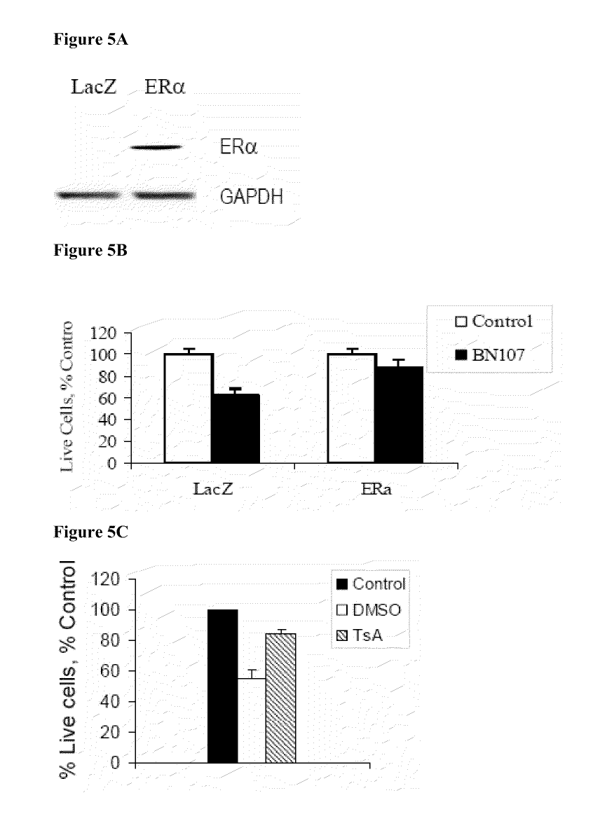

To ascertain that functional ER plays a protective role in BN107 induced apoptosis, ERα expression was transduced in MDA-MB-231 cells that are highly-sensitive to BN107 and are null for ER expression. FIG. 5A shows ERα protein expression after transduction and FIG. 2C shows WISP2 RNA expression, an ER responsive gene, indicating functional ER status. The ERα transduced cells and LacZ transduced control cells, were treated with BN107 for 18 hours and cell death was analyzed with AnnexinV PI binding. FIG. 5B shows that ERα expression in MDA-MB-231 cells significantly protected cells from BN107-induced apoptosis. Furthermore, ER− MDA-MB-231 cells were treated with trichostatin A (TsA), a differentiating agent, in attempt to reverse the mesenchymal phenotypes to re-express more epithelial markers. For example, MDA-MB-231 cells have been shown to re-express ERα, E-cadherin, and CD24, and down-regulate CD44, caveolin, and vimentin expression upon prolonged, low-dose TSA treatment. The levels of RNA expression of these genes have been examined in the MDA-MB-231 cells treated with TsA for 2 days, and confirmed the previous observations. These TsA differentiated MDA-MB-231 cells were then treated with BN107; and FIG. 2C shows that these cells conferred more resistance to BN107, consistent with the hypothesis that ERα status plays a protective role against BN107-induced apoptosis.

Major cellular pathways modulated by BN107 treatment—Reactive oxygen species (ROS) production or p38 activation induced by BN107 may not be the primary mechanism mediating the pro-apoptotic effect. In order to investigate the major underlying mechanism mediating the pro-apoptotic effect of BN107 in ER− breast cancer cells, expression array analysis was performed on Hs578T (ER−, sensitive) and MCF7 (ER+, insensitive) cells treated with BN107 for 4 hours (supplemental data). Expression profiles were analyzed using Ingenuity Pathway Analysis to identify potential cellular pathways collectively responsible for BN107 induced death. IPA analysis between these two cell lines revealed distinct patterns of gene expression in response to BN107. Specifically, ER− breast cancer cells responded to BN107 by up-regulating genes involved in cell death, oxidative stress response, MAPK signaling, and cholesterol synthesis/uptake pathways; while ER+ breast cancer cells did so by regulating a relatively small set of genes involved in growth receptor and survival signaling.

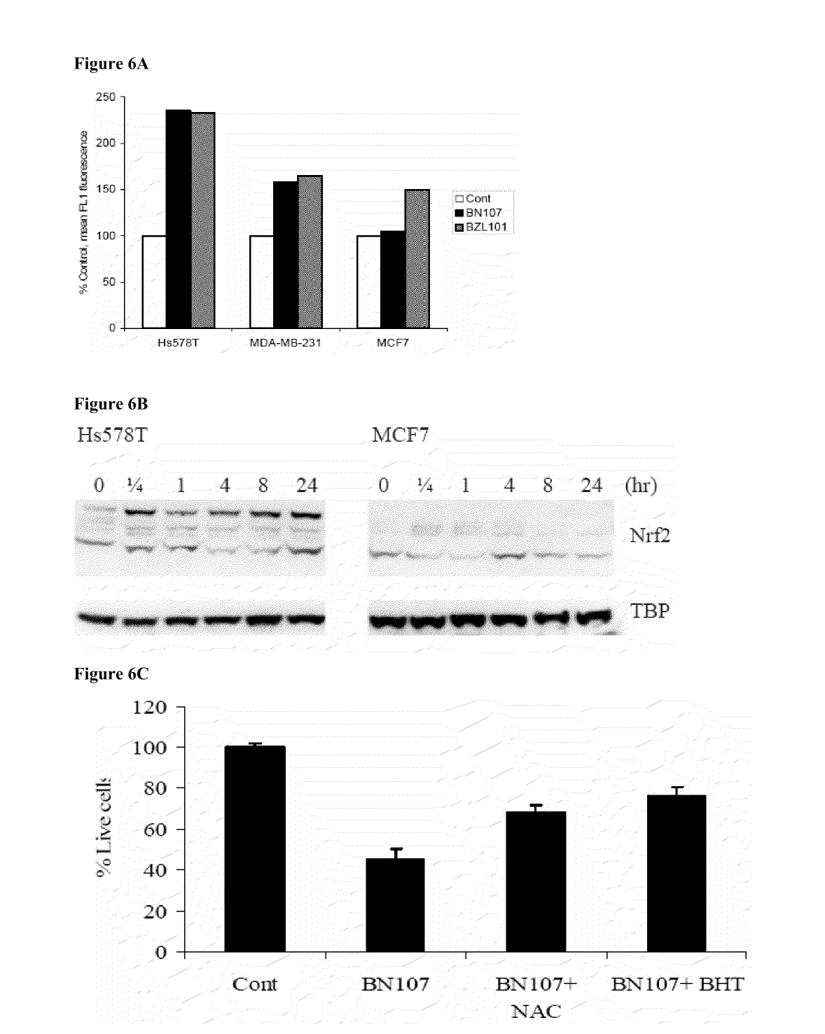

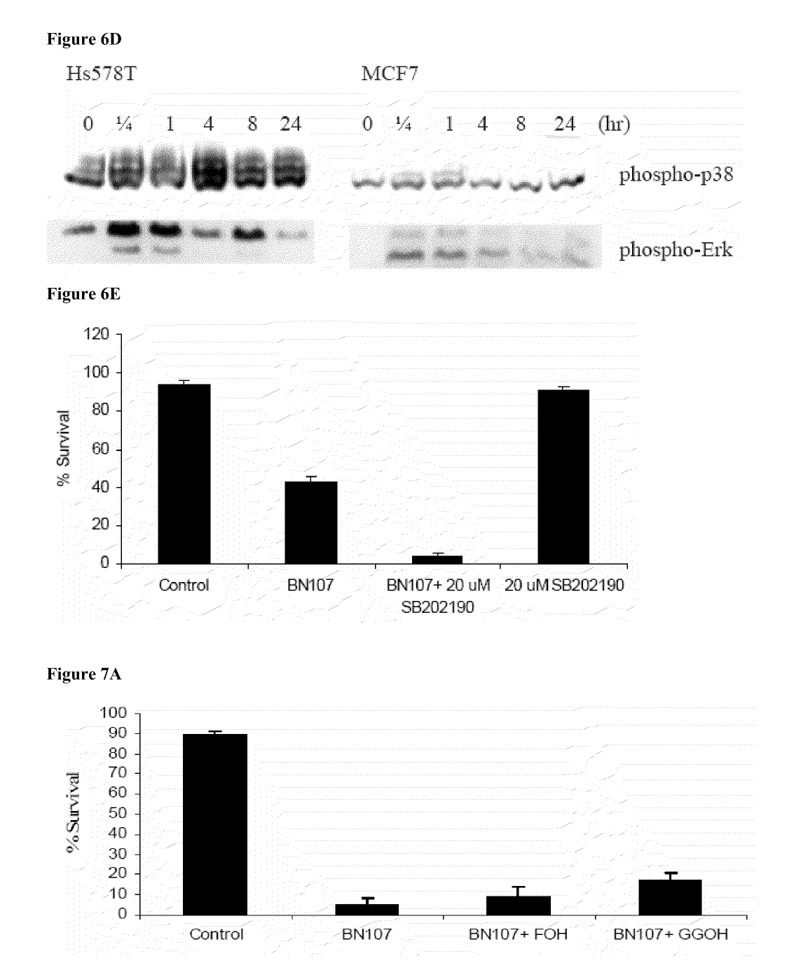

Since oxidative stress response was indicated in BN107-treated Hs578T cells, whether ROS production was induced and whether this could be causal to death by BN107 was examined. The cell-permeable ROS-sensitive probe CM-H2DCFDA was used, and showed that BN107 induced a significant accumulation of ROS in two sensitive breast cancer cell lines, Hs578T and MDA-MB-231; while it had no effect on MCF7 cells (FIG. 6A). BZL101 has been shown previously to induce strong ROS production and was used as a positive control. To further confirm that BN107 induces oxidative stress responses, the levels of Nrf2, a key transcription factor that translocates into nuclei in response to oxidative stress, were examined in BN107 treated cells. Western blot analysis showed a significant and sustained increase in nuclear Nrf2 levels in BN107 treated Hs578T cells and an insignificant and transient increase in MCF7 cells. However, the significant production of ROS did not lead to extensive oxidative DNA damage as measured by Comet assay or staining for 8-oxoguanine with Avidin-FITC. In addition, pre-incubation of cells with N-acetyl cysteine (NAC) or butylated hydroxytoluene (BHT), two strong ROS scavengers, only partially protected cells from BN107-induced apoptosis (FIG. 6B). These observations suggested that oxidative stress is not the primary cause of death induced by BN107.

Changes in MAPK signaling were confirmed by Western analysis. As shown in FIG. 6C, levels of phospho-Erk were rapidly induced in both Hs578T and MCF7 cells; while levels of phospho-p38 were only induced in the sensitive Hs578T cells. To determine if increased activity of p38 specifically in Hs578T cells was responsible for BN107-induced apoptosis, cells were pre-incubated with specific p38 antagonist SB202190 before treatment with BN107. As shown in FIG. 6D, p38 activation appeared to be a survival mechanism as co-treatment of p38 antagonist and BN107 produced synergistic cytotoxic effect.

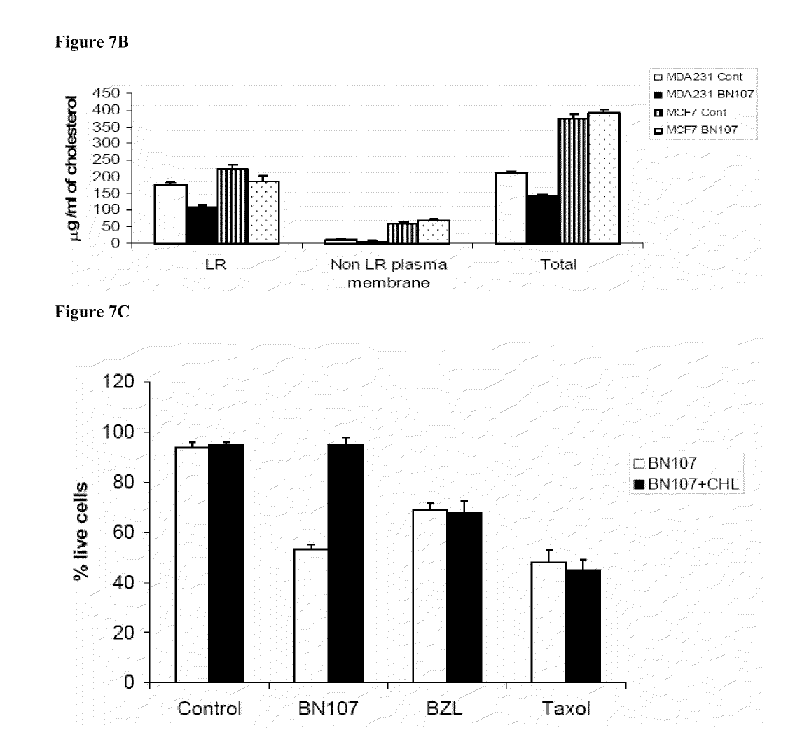

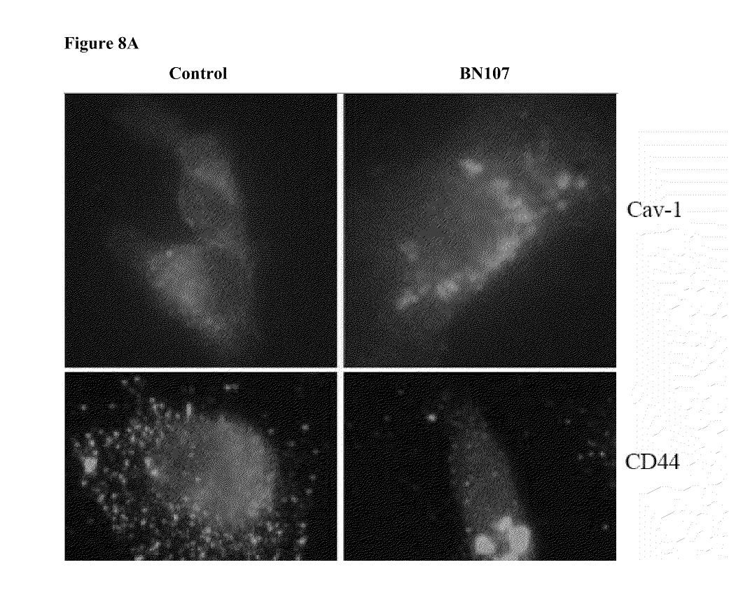

Cholesterol depletion induced by BN107 could potentially be responsible for its pro-apoptotic effect. Cholesterol synthetic/transport genes were also among the genes up-regulated in BN107 treated ER−Hs578T cells, suggesting that cholesterol or intermediates of cholesterol synthetic pathway might play a role in the pro-apoptotic effect of BN107. The cholesterol synthetic pathway provides isoprenoid precursors that are important for the functions of several signaling proteins essential for cell survival, such as RAS or RAS-related proteins. Isoprenylation of these proteins provides post-translational modification for their proper membrane localization and activities. Specifically, the farnesyl and geranylgeranyl moieties from farnesyl pyrophosphate and geranylgeranyl pyrophosphate are covalently linked to the C-terminus of RAS and RAS-related proteins. The corresponding alcohols for these pyrophosphates, farnesol (FOH) and geranylgeraniol (GGOH), restore cellular functions that have been altered by mevalonic acid depletion, a substrate in the cholesterol synthetic pathway. Therefore, it was postulated that providing cells with exogenously added isoprenoid precursors, FOH or GGOH, could rescue cells from BN107 induced death. As shown in FIG. 7A, pre-incubation of Hs578T cells with FOH or GGOH did not protect cells from BN107-induced apoptosis, implying that lack of isoprenoid precursors was not the underlying cause of death.

It has been shown that the oleanane saponins form complex with cholesterol and are capable of drawing cholesterol from the outer face of erythrocyte membranes. The levels of total cellular cholesterol were measured, and it was observed that there was a decline after 4 hours of treatment (FIG. 7B). It was hypothesized that the significant decline of total cellular cholesterol might be responsible for the pro-apoptotic effect of BN107. Next, ER−Hs578T or MDA-MB-231 cells were co-treated with cholesterol and BN107 and analyzed cell death after 18 hours of treatment. FIG. 7C shows that addition of cholesterol completely and specifically recued cells from BN107 induced death; while it had no effect on BZL101-, or taxol-induced death. Co-treatment of LDL and BN107 also completely abolished the pro-apoptotic effect of BN107. Of interesting note, addition of cholesterol into the media 2 hours after the treatment of BN107 also protected cells from death. These observations confirmed our hypothesis that cholesterol depletion was potentially the major underlying mechanism responsible for the pro-apoptotic effect of BN107.

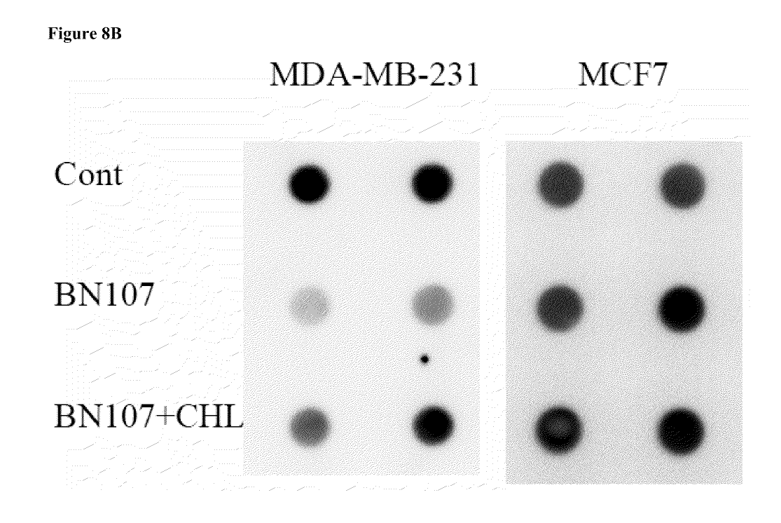

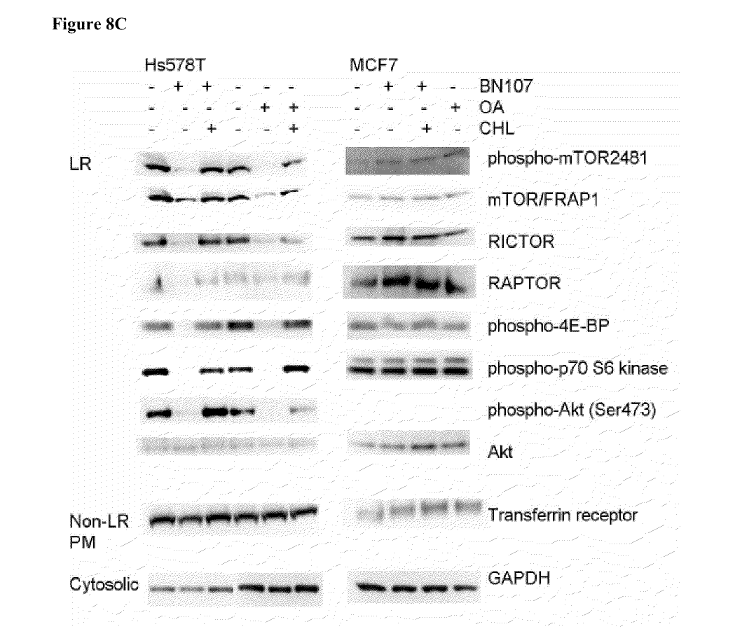

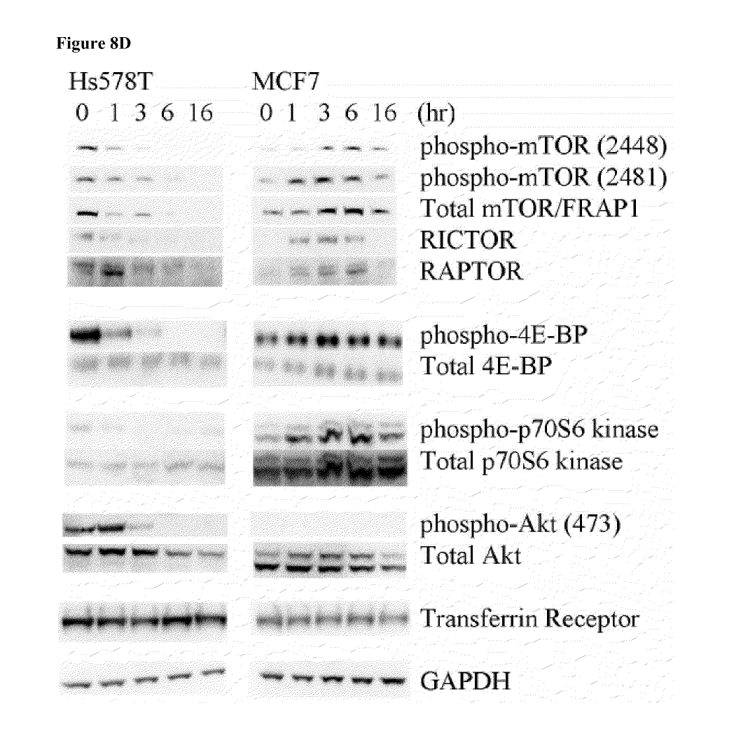

Lipid rafts were disrupted by BN107: Cholesterol is critically important for the functions of LRs, a specialized platform within plasma membrane that organizes signal transduction, including cell survival. Given the abundance of oleanane saponins present in BN107, it is therefore reasonable to hypothesize that BN107 strips/depletes membrane cholesterol which in turn disrupts the LRs-mediated survival signaling. The cholesterol levels in LRs were first measured; and FIG. 7B shows that the level of cholesterol in the lipid raft region was also depleted in the BN107-treated MDA-MB231 cells, consistent with the reduction in total level of cellular cholesterol. Next, the distribution pattern of caveolin and CD44, two LR resident proteins, were observed using immunofluorescent staining. FIG. 8A shows that BN107 treatment caused a rapid redistribution of these two LR resident proteins to intracellular, lysosomal-like localization within 4 hours. This observation was corroborated with data obtained using a biochemical subcellular fractionation approach. Specifically, the level of cytosolic caveolin protein increased and the level of plasma membrane caveolin protein reciprocally decreased after 4 hour of BN107 treatment; while total level of caveolin protein remained unchanged. These data prompted us to hypothesize that membrane LRs and LRs-mediated survival signaling might be disrupted by the BN107.

mTORC1 and mTORC2 components were displaced/degraded from lipid rafts leading to inhibition of mTORC1 and mTORC2 activities. To determine whether lipid rafts-mediated survival signaling was disrupted by BN107, the lipid raft fractions obtained from ultracentrifugation of triton-X100 solubilzed lysate were analyzed using sucrose gradient. The lipid raft region was identified with fractions enriched in gangliosides, GM-1, a marker for lipid raft region, in untreated cells. As shown in FIG. 8B, the level of GM-1 measured by dot blot analysis was significantly decreased in the LR fractions of BN107 treated MDA-MB-231 cells, thereby confirming ablation of rafts by BN107. Cholesterol replenishment reconstituted raft structures manifested by re-appearance of GM1 in the LR fractions. In BN107 resistant MCF7 cells, levels of GM-1 appeared to be unchanged.

As Akt/mTOR, the main survival signaling pathway, has been implicated to take place on LRs, it was hypothesized that BN107 and similarly OA disrupted the signaling of these complexes on LRs. The fractions collected from sucrose-density-centrifugation of BN107 or OA treated Hs578T or MCF7 lysates were dialyzed and concentrated. Same amounts of protein from LR (fractions 3-5), non-LR plasma membrane (f6), and cytosolic (f8) fractions were analyzed by Western blotting. The levels of RAPTOR, Akt, 4E-BP, p70S6 kinase were first observed in these collected fractions; and it was confirmed that they were all enriched in the LR fractions (data not shown). The levels of phospho-mTOR, total mTOR; as well as the mTORC1 and mTORC2 complex partner RAPTOR and RICTOR, respectively, were then measured in BN107 or OA treated Hs578T cells. All were significantly decreased in the LR fractions of Hs578T after 4 hours of treatment, indicating that the components of the mTORC1 and mTORC2 complexes were disrupted/displaced from this region. As the levels of mTOR/FRAP1 and RAPTOR protein decreased in the lipid raft region isolated from Hs578T cells treated with BN107 or OA, it was asked the question whether mTORC1 activity was inhibited. The activity of mTORC1 was determined by measuring the phosphorylation of its substrates, 4E-BP and p70S6 kinase (FIG. 8C); and it was found that indeed mTORC1 activity was greatly inhibited.

In addition, as the same pool of mTOR/FRAP1 polypeptide is shared between the mTORC1 and mTORC2 complex and the mTORC2 complex partner RICTOR was decreased in the lipid rafts of BN107 or OA-treated Hs578T cells, it was hypothesized that mTORC2 activity would be inhibited as well. Given the recent discovery that mTORC2 is the main kinase phosphorylating Akt at Ser473, and Akt signaling has been implicated to take place on LRs, the level of Ser473 phosphorylated Akt was analyzed in LR fractions as a read-out for mTORC2 activity. In FIG. 8C, it is shown that BN107 or OA treatment decreased the level of Ser473-phosphorylated Akt, while they had no effect on the total level of Akt on LRs. These data suggested that upstream regulator of Akt was disrupted in LRs, likely to be the mTORC2 complex shown to have less total mTOR/FRAP1 and RICTOR components. Conversely, addition of exogenous cholesterol restored these signaling events disrupted by BN107 or OA (FIG. 8C). None of these changes were observed in the resistant MCF7 cells (panel on the right, FIG. 8C). The levels of transferrin receptor (TR) marking the non-LR plasma membrane region and GAPDH marking the cytosolic fractions were not affected in both cell lines by either BN107 or OA treatment.

To ascertain that these signaling changes occurred at LRs indeed translated into the whole-cell level. FIG. 8D shows the total levels of these signaling proteins. Consistent with the data shown within the lipid raft fractions, levels of total mTOR/FRAP1, phospho-mTOR, RAPTOR, and RICTOR were all decreased within one hour of BN107 treatment, resulting in minimal mTORC1 and mTORC2 activities to phosphorylate 4E-BP and p70S6 kinase, and Ser473-Akt, respectively. The decrease in total protein levels of mTOR/FRAP1, RAPTOR and RICTOR occurred post-transcriptionally as the levels of their corresponding mRNAs were not modulated by BN107 or OA treatment in the sensitive ER− Hs578T cells (data not shown). Also consistent with levels on LRs, Ser-473 phosphorylated Akt was dramatically decreased; while levels of total cellular Akt remained unchanged. These data collectively indicated that LRs and LRs-mediated growth/survival signaling were specifically disrupted by BN107 and OA. These effects were presumably due to their cholesterol binding/stripping effect on LRs, as addition of exogenous cholesterol seemed to reverse these events (FIG. 8 C).

### Discussion

Identifying molecular targets for aggressive types of breast cancer is a milestone in the pursuit of individualized therapies. Gene-expression profiling of primary tumors has led to the following subcategories: luminal A, luminal B, the human epidermal growth factor receptor 2 (HER2) and the basal-like subtypes. Approximately 16% of all breast cancers are basal/mesenchymal like and these tumors do not respond to available targeted therapies and patients often die within two years of diagnosis. What sets these tumors apart is that unlike many breast cancers, basal/mesenchymal-like tumors are less differentiated, and more aggressive in general which do not express the ER or PR, nor do they have amplified HER2, referred to as ‘triple negative’ breast cancer. Women with triple negative tumors are not eligible for treatments that target ER (tamoxifen, aromatase inhibitors) or HER2 (trastuzumab). Instead they are treated with conventional chemotherapies, which have limited efficacy and many side effects. Therefore, it is critically important to identify alternative therapeutic strategies for these patients. In this study it was demonstrated that BN107 and its predominant oleanane saponins, oleanolic acid, target specifically the mesenchymal-like, ER− breast cancer cells; while the ER expressing cells are not sensitive to these treatments. When the publicly available expression profiles of various breast lines were clustered according to their sensitivity to BN107, it was found that expression of many ER down-stream targets were associated with insensitivity to BN107. The pro-apoptotic effect induced by BN107 or OA did not correlate with Her2 or EGFR status (Table 2-1). It was further shown that when ER status was restored in breast cancer cells lacking functional ER by forced expression with adenovirus or induced expression with deacetylating agent TsA, the sensitivity to BN107 in these cells were significantly decreased. Collectively, these findings demonstrated that functional ER status played a protective role in BN107-induced apoptosis and suggested the possibility of developing BN107− or oleanane saponins-based therapeutic strategies for the triple negative breast cancer patient population.

To elucidate the mechanism mediating the selective pro-apoptotic effect on ER− breast cancer cells elicited by BN107, expression profiling analysis was performed, comparing and contrasting expression patterns in the sensitive (ER−) V.S. the insensitive (ER+) cell lines. In the sensitive line, gene expression patterns consistent with cell death, oxidative stress, MAPK signaling transduction, and cholesterol synthetic/uptake pathway were identified. Indeed, it was shown that oxidative stress was induced while only partially contributed to BN107-induced death. The other significant cluster of genes identified was activation of MAPK signaling pathway. It was confirmed that p38 was selectively activated in the ER− sensitive line in response to BN107 treatment. However, p38 activation was presumed to be a survival mechanism because blocking p38 activation lead to synergistic death with BN107 treatment. Finally, it was shown that depletion of lipid raft cholesterol, not isoprenoid precursors, appeared to be responsible for BN107− induced apoptosis, as supplying exogenous cholesterol or cholesterol equivalents (i.e. LDL) protected cells specifically and completely from death induced by BN107 and OA. The integrity of lipid rafts is very dependent upon the presence of cholesterol. The loss of cholesterol from lipid raft by treatment with cholesterol-sequestering agents (methyl-β-cyclodextrin, MβCD), through increased sterol, or by inhibiting its de novo synthesis leads to loss of raft-associated proteins and decreased cell survival.

It was further showed that BN107 or oleanolic acid-induced apoptosis was based on their abilities to inhibit the survival signaling events, namely Akt/mTOR pathway, that take place on the cholesterol-rich LRs. Aberrant activities of Akt/mTOR pathway have been shown to exist in many cancers, which allow the malignant tumor cells to proliferate and evade death signaling or become resistance to various therapies. Despite numerous efforts have been directed to develop therapeutics targeting mTOR activities, most clinical testing has not shown promising results against solid tumor cancers. The unimpressive data of these mTOR inhibitors, namely rapamycin and its analogs, points to our incomplete understanding of the regulation of mTOR pathways, especially involving mTORC2 activity. Recent data have implicated mTORC2 activity as the major kinase that phosphorylates Ser473 on Akt, along with PDK1, facilitate the full activation of Akt. Rapamycin showed minimal acute inhibitory effect on mTORC2, as compared to mTORC1, albeit prolong incubation lead to some levels of inhibition on mTORC2. mTORC1 inhibition alone by rapamycin can lead to PI3K/Akt pathway reactivation. Conversely, disruption of mTORC2 activity along might also lead to increase in mTORC1 activity. Altogether, these observations would suggest that pathway activation and reactivation could be avoided by agents that lead to concomitant Akt and mTOR inhibition (that would target both mTORC1 and mTORC2). Here, it is reported that BN107 and OA selectively decreased mTORC1 and mTORC2 activities in the LRs of ER− breast cancer cells, which presumably led to concomitant inhibition of Akt activity. Although the possibility cannot be ruled out that a decrease in the level of [P1 (4,5)P2] in LRs as a result of cholesterol depletion might lead to reduction of Akt membrane recruitment and phosphorylation.

The inhibition of mTORC1 and mTORC2 activities appeared to be based on disruption of the mTORC1 and mTORC2 complex formation on lipid rafts (LR). This was due to less amounts of the complex components present on LRs, namely mTOR/FRAP1, RAPTOR and RICTOR. The disruption of the complexes likely led to degradation of these proteins, as the total cellular levels of them also showed concomitant decrease. The decrease in the total protein levels of mTOR/FRAP, RAPTOR, and RAPTOR was not a result of down-regulation in their steady-state RNA levels (data not shown). Although the possibility cannot be ruled out that BN107 or OA specifically down-regulated the levels of these proteins post-transcriptionally, which resulted in decreased mTORC1 and mTORC2 activities on LR. Aside from reports that farnesylthiosalicylic acid and curcumin could inhibit mTORC1 activity by dissociating the mTOR/FRAP1-RAPTOR complex, as best understood, this is the first report demonstrating the possibility of regulating the activity of mTOR complexes by treating cells with agents that decrease the levels of mTORCs components on LRs, as well as total cellular level.

Cholesterol has been associated with tumor progression. Experimental and epidemiological evidence suggests that cholesterol may play a promotional role in cancer development and progression. It has been proposed that progressive increases in membrane cholesterol contribute to the expansion of rafts, which may potentiate oncogenic pathways (for example, Akt) of cell signaling. These findings collectively suggest that agents interfering with cholesterol homeostasis in LRs, such as BN107 and OA, represent a novel approach to disrupt tumor cell survival signaling.

A major concern connected to the potential clinical application of raft-ablating chemicals is that these agents may also non-selectively alter LRs and interfere with function in cells of vital organs like heart, liver, kidney, pancreas, etc. Completely opposite to this notion, MβCD derivatives are widely utilized as carriers for water-insoluble drugs for parenteral use, implying that lower doses of these compounds do not ultimately exert marked systemic toxicity. Albeit Gleditsia saponins have been shown to strip plasma membrane cholesterol from erythrocytes in vitro, anti-tumor doses of OA have been shown to exhibit minimal toxicity in animals. It must be noted, as well, that distinct types of LRs have been identified that differ in their biochemical composition, compartmentalization and functions. Indeed, many studies have shown that depletion of cholesterol from cells leads to the disruption of LRs and the release of raft constituents into the bulk plasma membrane. However, not all LRs appear to be equally sensitive to cholesterol depletion. For example, depletion of cholesterol from enterocyte explants by treatment with MβCD removed 70% of the microvillar cholesterol, but did not affect the ability of a raft marker protein, galectin-4, to localize to the low-density triton X-100-insoluble membrane fractions. Similarly, Rajendran et al showed that, in Jurkat cells and U937 cells, several raft proteins including lck, lyn and LAT were released from rafts by treatment with MβCD, but flotillins remained in low-density detergent-resistant domains. These findings suggest that there is heterogeneity in the LRs population in terms of its dependence on or interaction with cholesterol. Consistent with this notion, Ostapkowicz et. al. showed that lipid rafts undergo significant structural reorganization during transition from ER+ (i.e.MCF7) breast cancer cells to the more invasive (MDA-MB-231) breast cancer. It is, therefore, possible that only a specific subset or composition of LRs supports Akt/mTOR signaling that was inhibited by BN107 or OA in the ER− breast cancer cells. However, how ERα contributes to the protection of BN107-induced lipid raft disruption and apoptosis is entirely unknown and is under investigation. Further detailed characterization of the specific interactions between BN107/OA and various LRs components that will facilitate development of drugs selectively targeting raft components associated with Akt/mTOR signaling appears critical at this stage.

### Materials & Methods

Reagents and Antibodies

BN107 is an aqueous preparation of the grounded fruit of Gleditsia senensis (Sichuan Medicines and Health Products, Chengdu, China Campbell's paper). Briefly, 10 grams of grounded powder was weighed out and added to 100 ml of distilled water. The herbal mixture was brought to boil with constant stirring. Once reaching boiling point, the heat was reduced to maintain temperature at 70° C. and simmered for additional 40 minutes. The herbal mixture was then taken off the hot plate and cooled down to 50° C. before it was centrifuged at 3000 RPM for 20 minutes at 4° C. The supernatant was decanted into a new tube and centrifuged for another 20 minutes. The supernatant was aliquoted and stored at −80° C. for future use. One ml of supernatant was freeze-dried over night to determine yield (typical yield, 50-55 mg/ml). New batch was generated every 3 months to ensure no activity loss. OA is dissolved in DMSO and cholesterol is dissolved in 100% ethanol.

All chemicals were purchased from Sigma unless noted otherwise. CM-H2DCFDA was purchased from Invitrogen. The following antibodies were purchased from Cell Signal except noted: phospho-mTOR, total mTOR, RICTOR, RAPTOR, phosho-4EBP, total 4EBP, pS6 kinase, total S6 kinase, phospho-AKT, total AKT (Santa Cruz), Nrf2 (Santa Cruz), Caveolin 1 (BD Biosciences), TBP (Abeam), CD44 (Epitomics), ERa ( ) and cytochrome C (Biovision). GM-1 was detected by using subunits of CT subunit B conjugated to HRP (Invitrogen).

Cell Cultures and Treatments

All the cell lines used were purchased from ATCC. Cells were treated with 70 μg/ml of BN107 (calculated based on freeze-dry weight). The dose is determined based on the EC50 of each batch in killing 50% of Hs578T or MDA-MB-231 cells 18 hours after treatment. The cells were treated with 110 μM (EC50 for Hs578T) or 125 μM (EC50 for MDA-MB-231) of OA for various time points as indicated for different assays.

ERα Transduction

Sixty percent confluent MDA-MB-231 cells were transduced with adenovirus particles expressing LacZ or ERa in the presence of 4 μg/ml polybrene on day 0. Infected cells (300,000) were trypsinized and plated in the presence of 10 nM estradiol per well in 6-well plate on Day 1 AM. Treatment of these cells were started on Day1 PM and continued for 16 hours.

Cell Death/Apoptosis Measurement

Cell survival was measured using FACS analysis of AnnexinV-alexa 488/PI bound cells following the manufacturer's instruction (Invitrogen). Mitochondrial transmembrane potential (MTP) was determined using JC-1 dye (Invitrogen) in live cells and analyzed using flow cytomery. Caspase 3 and 9 activities were measured using specific caspase peptide inhibitors (Calbiochem) conjugated to FITC followed by flow cytometry analysis. Cytosolic cytochrome C release were determined by separating the cytosol from mitochondria (Biovision) and cytochrome C released in cytosol was shown using immunoblotting.

ROSCells were treated with BN107 or BZL101 (strong ROS inducer) for 15 minutes before loading with CM-H2DCFDA.

Immunostaining

Twenty thousands MDAMB-231 cells were plated in one well of 8-chamber slides on day 0. The cells were treated with 70 μg/ml of BN107 for 4 hours and fixed with either cold 4% paraformaldehyde in PBS for 10 minutes or methanol:acetone (1:1) at −20° C. for 5 minutes. Cells were rinsed in PBS and blocked in 2% BSA in PBS for one hour before applying anti-caveolin (1/1000) or anti-CD44 (1:250) in 2% BSA in PBS overnight at 4° C. The chamber slides were rinsed with PBS and incubated with appropriate Alexa 488 conjugated secondary antibody for one hour. The nuclei were stained with 1 μg/ml Hoechst 33258 for 5 minutes and the slides were mounted with Fluoromount-G (SouthernBiotech) before viewing.

Cholesterol Content Determination

Total cellular cholesterol levels were determined by lysing cells in RIPA buffer and extracted using chloroform (3 times). Cholesterol content in lipid raft region were determined by using fractions enriched with GM-1, as determined by dot blot analysis of fractions collected after cellular fractionation using sucrose or Nycodenz gradients (yielding similar results). Fractions positive for GM-1 expression were subjected to chloroform extraction (3 times). The pooled organic phase were dried down and subjected to vacuum. The Amplex Red cholesterol assay kit was used to quantitate the amount of cholesterol and cholesterol ester in the samples (Invitrogen).

Lipid Raft Isolation

A modified procedure for density gradient centrifugation using Nycodenz from Sigma-Aldrich (St. Louis, Mo.) was used to fractionate Triton X-100-soluble and Triton X-100-insoluble membrane and cytoskeletal subdomains and complexes. Cell lysates were prepared by mixing equal volumes of cell pellets with 2% Triton X-100 on ice for 1 minute and subsequent dilution with equal volume of PBS. The resulting lysate (3-4 mg protein) were incubated on ice for 5 minutes and further diluted with equal volume of 35% Nycodenz [5′-(N-2,3-dihydroxypropylacetamido)-2,4,6-triiodo-N,N-bis(2,3-dihydroxypropyl)-isophtalamide] in PBS to achieve 17.5% Nycodenz final concentration. Density step gradient was generated by applying 0.5 mL aliquots of increasing concentration of Nycodenz (35%, 25%, 22.5%, 20%, lysate in 17.5%, 15%, 12%,8%, and 4%) sequentially into Beckman (Palo Alto, Calif.) 13×51 mm polyallomer tubes. Lysates were placed in the middle of Nycodenz gradient premixed in 17.5% Nycodenz. Tubes were centrifuged at 46,000 rpm for 16 hours in a Beckman 55 Ti rotor at 4° C. Following centrifugation, 0.5 mL fractions were carefully withdrawn and small pellet was resuspended in PBS containing 0.5% SDS and 1% Triton X-100 (fraction 10). Total of 10 fractions and control input lysate were analyzed for the distribution of proteins by Western blot. Typically, components of light lipid rafts were distributed into first three to five fractions (as marked by GM-1), non-lipid raft cell membrane components were distributed in fraction 6 (as marked by transferring receptor); soluble cell components, including cytosolic proteins, remained in fractions 7, and 8 and cytoskeleton-associated high-density fractions were distributed in fractions 9,10. The fractions were dialyzed against PBS to remove the gradient sugars and concentrated using Amicon Ultra 4 centrifugal filter device (Millipore) before protein quantitation (BCA reagent, Thermo Fisher). The expression of GM1 in fractions was tested using horseradish peroxidase-conjugated cholera toxin B subunit (Invitrogen) and dot blot analysis.

Western Blotting

Cell extracts, obtained by scraping cells in PBS in the presence of protein phosphatase and protease inhibitors and lysing with ice-cold RIPA buffer, were loaded on the SDS-PAGE at 25-30 μg per lane. Same amounts of proteins (25 μg) were precipitated from lipid raft fractions and loaded onto 3-7% TA gel or 4-12% Bis-Tris gels. Separated proteins were transferred onto nitrocellulose membrane (iBlot, Invitrogen) and used for probing with specific antibodies following manufacturer's instruction. Blots were reused several times after mild stripping (Restore, ThermoFisher) when necessary. Secondary antibodies conjugated to horseradish peroxidase and SuperSignal West Dura Extended Duration substrates were used to develop images on Kodak imager.

TABLE 2-1
Table 2-1: Cells without ER are more sensitive to BN107 induced apoptosis,
while Her2 status appears not correlative with BN107 sensitivity.

| | Annexin V PI | | |
|---|---|---|---|
| | staining | ER | Her2 |
| SKBr3 | ++ | − | + |
| Hs578T | ++++ | − | + |
| MDA-MB-468 | ++ | − | − |
| MDA-MB-231 | ++++ | − | − |
| MDA-MB-453 | ++++ | − | + |
| MCF10A | +++ | − | − |
| IMR90 | ++++ | − | − |
| MDA-MB-361 | −/+ | + | + |
| BT474 | −/+ | + | + |
| MCF7 | − | + | − |

Cells were treated with BN107 and harvested after 18 hours for analysis of Annexin V/PI binding. The summary shown is a result of 3 independent experiments.

### Example 3

### Open Label Increasing Dose, Dosing Study

In order to assess the safety and maximum tolerated dose (MTD) of an extract of Gleditsia sinensis Lam (Study Drug), the following protocol is carried out.

Study Drug comprises 1 mg (week 1), 10 mg (week 2), 100 mg (week 3) or 1000 mg (week 4) of extract of Gleditsia sinensis Lam in suitably sized gelatin capsules or dissolved in water. (Hereinafter the extract of Gleditsia sinensis Lam may be referred to as “Study Drug”). The dose may be split between two or more gelatin capsules if necessary, and/or may be administered q.d. or b.i.d., optionally as a tea. Normal, healthy volunteers of age 18 to 60 are administered 1 mg per day of Study Drug for week 1, 10 mg per day of Study Drug for week 2, 100 mg per day of study drug for week 3 and 1000 mg per day of Study Drug for week 4. Subjects are monitored for appearance of any adverse events. At any time, if a subject appears to not tolerate the current dose, the attending medical staff will note such intolerance. The maximum tolerated dose will be considered the highest dose at which each of the subjects tolerates the dose, or, if no subject experiences intolerance, 1000 mg of the Study Drug per day.

### Example 4

### Dose Escalation Study

In order to assess the safety and maximum tolerated dose (MTD) of an extract of Gleditsia sinensis Lam (Study Drug), the higher or tighter dosage ranges of extract of Gleditsia sinensis Lam are administered to a suitable patient population, such as a patient population having identifiable ER negative breast cancer, PR negative breast cancer, Her2/neu negative breast cancer and/or triple negative breast cancer. One or more of the patients selected are characterized by prior, unsuccessful treatment for cancer. One or more additional dosage ranges, such as a dose between 100 mg and 1000 mg, or a dose between 1000 mg and 10 grams, or a dose between 10 grams and 1000 grams, is chosen to evaluate the therapeutic index of the drug and its maximum therapeutic dose. Dosage that may be evaluated include 500 mg, 1000 mg, 10 grams, 20 grams, 30 grams, 40 grams, 50 grams, 60 grams, 75 grams and 100 grams dry weight of Gleditsia sinensis Lam.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a patient having estrogen receptor (ER) negative breast cancer, comprising administering to the patient a therapeutically effective amount of an agent selected from the group consisting of an extract of Gleditsia sinensis Lam, oleanolic acid, a pharmaceutically acceptable salt of oleanolic acid, and a combination of two or more thereof.

2. The method of claim 1, wherein the therapeutically effective amount of the agent is about 0.001 to about 100 grams dry weight of the agent per day.

3. The method of claim 1, wherein the ER negative breast cancer is also negative for one or both of progesterone receptor (PR) and/or Her2/neu.

4. The method of claim 1, wherein the ER negative breast cancer is triple negative breast cancer.

5. The method of claim 1, wherein the ER negative breast cancer is metastatic.

6. The method of claim 1, wherein the agent is in an oral dosage form.

7. A pharmaceutical composition comprising a therapeutically effective amount of an agent selected from the group consisting of an extract of Gleditsia sinensis Lam, oleanolic acid, a pharmaceutically acceptable salt of oleanolic acid, and combinations of two or more thereof, wherein the therapeutically effective amount is an amount effective to treat estrogen receptor (ER) negative breast cancer.

8. The pharmaceutical composition of claim 7, wherein the therapeutically effective amount of the agent is about 0.001 to about 100 grams dry weight of the agent per day.

9. The pharmaceutical composition of claim 7, wherein the ER negative breast cancer is also negative for one or both of progesterone receptor (PR) and/or Her2/neu.

10. The pharmaceutical composition of claim 7, wherein the ER negative cancer is triple negative breast cancer.

11. The pharmaceutical composition of claim 7, wherein the cancer is metastatic.

12. The pharmaceutical composition of claim 7, wherein the extract of Gleditsia sinensis Lam is in an oral dosage form.

13. A method of treating a patient having cancer that does not express an estrogen receptor (ER), comprising administering a therapeutically effective amount of an agent selected from the group consisting of an extract of Gleditsia sinensis Lam, oleanolic acid, a pharmaceutically acceptable salt of oleanolic acid, and combinations of two or more thereof.

14. The method of claim 13, wherein the therapeutically effective amount of the agent is about 0.001 to about 100 grams dry weight of the agent per day.

15. The method of claim 13, wherein the agent is in an oral dosage form.

16. The method of claim 13, wherein the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

17. A pharmaceutical composition comprising a therapeutically effective amount of an agent selected from the group consisting of an extract of Gleditsia sinensis Lam, oleanolic acid, or a pharmaceutically acceptable salt of oleanolic acid, wherein the therapeutically effective amount of the agent is an about that is effective to treat a cancer that does not express an estrogen receptor (ER).

18. The pharmaceutical composition of claim 17, wherein the therapeutically effective amount of the agent is about 0.001 to about 100 grams dry weight of the agent per day.

19. The pharmaceutical composition of claim 17, wherein the agent is in an oral dosage form.

20. The pharmaceutical composition of claim 17, wherein the cancer that does not express the ER is selected from the group consisting of: bone cancer, brain stem glioma, breast cancer, cancer of the adrenal gland, cancer of the anal region, cancer of the bladder, cancer of the endocrine system, cancer of the esophagus, cancer of the head or neck, cancer of the kidney, cancer of the ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, a skin cancer, spinal axis tumors, stomach cancer, uterine cancer, and combinations thereof.

21. A method of treating a patient having estrogen receptor (ER) negative breast cancer, comprising administering a therapeutically effective amount of at least one saponin, or a pharmaceutically acceptable salt thereof, to the patient, wherein the saponin possesses mTORC1, mTORC2, and/or possesses Akt inhibitory activity, and/or disrupts lipid rafts (LRs) in vitro.

22. The method of claim 21, wherein the saponin possesses mTORC1 and mTORC2 activity, the saponin possesses Akt inhibitory activity, and the saponin disrupts lipid rafts.

23. The method of claim 21, wherein the therapeutically effective amount of the saponin is about 0.001 to about 100 grams dry weight per day.

24. A pharmaceutical composition comprising a therapeutically effective amount of at least one saponin, or a pharmaceutically acceptable salt thereof, wherein the saponin possesses mTORC1, mTORC2, and/or possesses Akt inhibitory activity, and/or disrupts lipid rafts (LRs) in vitro.

25. The pharmaceutical composition of claim 24, wherein the saponin possesses mTORC1 and mTORC2, the saponin possesses Akt inhibitory activity, and the saponin disrupts lipid rafts.

26. The pharmaceutical composition of claim 24, wherein the therapeutically effective amount of the saponin is about 0.001 to about 100 grams dry weight of the saponin per day.

27. A method of treating a patient having cancer that does not express an estrogen receptor (ER), comprising administering a therapeutically effective amount of a saponin to the patient, wherein the saponin possesses mTORC1, mTORC2, and/or possesses Akt inhibitory activity, and/or disrupts lipid rafts (LRs) in vitro.

28. The method of claim 27, wherein the saponin possesses mTORC1 and mTORC2 activity, the saponin possesses Akt inhibitory activity, and the saponin disrupts lipid rafts.

29. The method of claim 27, wherein the therapeutically effective amount of the saponin is about 0.001 to about 100 grams dry weight of the saponin per day.

30. The method of claim 27, wherein the saponin, or pharmaceutically acceptable salt or derivative thereof, is in an oral dosage form.
